(19) United States
(12) Patent Application Publication
(10) Pub. No.: US 2017/0145002 A1
(43) Pub. Date: May 25, 2017

(54) DEUTERATED ISOXAZOLE DERIVATIVES AND THEIR USE AS METABOTROPIC GLUTAMATE RECEPTOR POTENTIATORS

(71) Applicant: AstraZeneca AB, Sodertalje (SE)

(72) Inventor: Mark E. Duggan, Tequesta, FL (US)

(21) Appl. No.: 15/358,629

(22) Filed: Nov. 22, 2016

Related U.S. Application Data
(60) Provisional application No. 62/260,148, filed on Nov. 25, 2015.

(51) Int. Cl. C07D 413/04

(52) U.S. Cl. CPC C07D 413/04

(57) ABSTRACT

The present application relates to a compound of Formula (I), and to uses thereof, such as for the treatment of a neurological or psychoartic disorder.

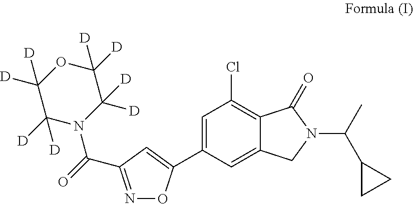 [SMILES: [2H]C1([2H])OC([2H])([2H])C([2H])([2H])N(C(=O)c2cc(-c3cc(Cl)c4c(c3)CN(C(C)C3CC3)C4=O)on2)C1([2H])[2H]]

### RELATED APPLICATIONS

This application claims the benefit of U.S. Provisional Application No. 62/260,148, filed Nov. 25, 2015, which is incorporated by reference herein in its entirety.

### BACKGROUND OF THE DISCLOSURE

The metabotropic glutamate receptors (mGluR) constitute a family of GTP-binding protein (G-protein) coupled receptors that are activated by glutamate, and have important roles in synaptic activity in the central nervous system, including neural plasticity, neural development and neurodegeneration. mGluR family receptors are implicated in a number of normal processes in the mammalian CNS, and are important targets for compounds for the treatment of a variety of neurological and psychiatric disorders. More recently, studies have provided evidence that certain mGlu receptor subtypes can regulate various addiction-related behaviors (WIREs Membr Transp Signal 2012, 1:281-295 and references therein). For instance, pharmacological studies have shown that mGlu5 receptors are involved in the acquisition and/or expression of the conditioned rewarding effects of cocaine, heroin, nicotine, amphetamines, morphine, and alcohol (WIREs Membr Transp Signal 2012, 1:281-295 and references therein). Studies have also shown that mGlu5 receptor antagonists may help reduce self-administration of cocaine, nicotine, or alcohol. Other studies have shown that mGlu2/3 agonists can suppress self-administration of cocaine, nicotine, and ethanol (Biological Psychiatry Oct. 1, 2015; 78:452-462).

The World Health Organization currently estimates that 1 billion people worldwide smoke. In 2013, an estimated 42.1 million people in the United States smoked cigarettes (17.8% of all adults; Centers for Disease Control and Prevention website, October 2015). The habit of smoking is a function of operant conditioning (Covino and Bottari, Journal of Dental Education 2001; 65:340-347). Millions of smokers worldwide are trying to quit. Tobacco/nicotine dependence is a condition that often requires repeated intervention (Centers for Disease Control and Prevention website, October 2015). Current strategies for smoking cessation include nicotine replacement therapy (NRT) patches and gum, nicotinic receptor partial agonists like Chantix, and certain antagonists of neuronal nicotinic acetylcholine receptors (nACh receptors) like bupropion. However, current smoking cessation drugs present significant disadvantages, and better treatments with fewer side effects are needed.

### SUMMARY OF THE DISCLOSURE

The present application provides deuterated compounds that exhibit activity as modulators of metabotropic glutamate receptors.

The present application provides a compound of formula (I),

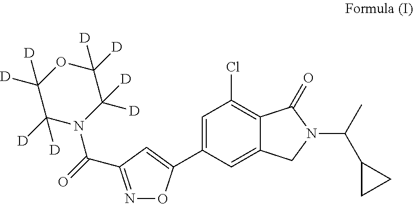 [SMILES: [2H]C1([2H])OC([2H])([2H])C([2H])([2H])N(C(=O)c2cc(-c3cc(Cl)c4c(c3)CN(C(C)C3CC3)C4=O)on2)C1([2H])[2H]]

or an enantiomer thereof.

In certain embodiments, the compound is (S)-7-chloro-2-(1-cyclopropylethyl)-5-(3-(morpholine-4-carbonyl-2,2,3,3,5,5,6,6-d₈)isoxazol-5-yl)isoindolin-1-one, represented by Formula (II):

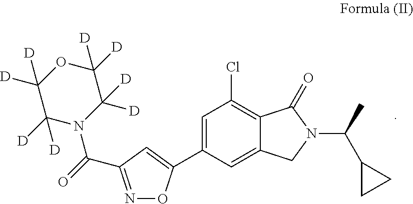 [SMILES: [2H]C1([2H])OC([2H])([2H])C([2H])([2H])N(C(=O)c2cc(-c3cc(Cl)c4c(c3)CN([C@@H](C)C3CC3)C4=O)on2)C1([2H])[2H]]

In certain embodiments, the application relates to the use of a compound of formula (I) or (II), or an enantiomer thereof, for the treatment of a neurological or psychiatric disorder associated with glutamate dysfunction.

In certain embodiments, the application relates to the use of a compound of formula (I) or (II), or an enantiomer thereof, in the manufacture of a medicament for the treatment of a neurological or psychiatric disorder associated with glutamate dysfunction.

In some embodiments, the neurological or psychiatric disorder is selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance disorder, substance use disorder, substance withdrawal disorder, substance abuse disorder, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, and post-traumatic pain, tardive dyskinesia, sleep disorders, narcolepsy, attention deficit/hyperactivity disorder, and conduct disorder.

In some embodiments, the neurological or psychiatric disorder is a substance use disorder. In some embodiments, the substance use disorder comprises a substance withdrawal disorder or a substance abuse disorder. In certain such embodiments, the substance is nicotine.

In some embodiments, the substance use disorder is a substance abuse disorder. In certain such embodiments, the substance is nicotine.

In some embodiments, the neurological or psychiatric disorder is a substance abuse disorder. In certain such embodiments, the substance is nicotine.

In certain embodiments, the substance abuse disorder is an alcohol abuse disorder, a tobacco products abuse disorder, a cannabis abuse disorder, a stimulant abuse disorder, a depressant abuse disorder, a hallucinogen abuse disorder, or an opioid abuse disorder. In certain such embodiments, the substance is nicotine.

In certain embodiments, the substance abuse disorder is a tobacco products abuse disorder. In certain such embodiments, the substance is nicotine.

In certain embodiments, the substance use disorder is a substance withdrawal disorder. In certain such embodiments, the substance is nicotine.

In certain embodiments, the neurological or psychiatric disorder is a substance withdrawal disorder. In certain embodiments, the substance withdrawal disorder is a tobacco products withdrawal disorder. In certain such embodiments, the substance is nicotine.

In certain embodiments, the treatment is directed towards smoking cessation.

In certain embodiments, the application relates to a pharmaceutical composition comprising a compound of formula (I) or (II), or an enantiomer thereof, and at least one pharmaceutically acceptable carrier, excipient, or diluent.

In certain embodiments, the application relates to the use of the pharmaceutical composition for the treatment of a neurological and psychiatric disorder associated with glutamate dysfunction.

In certain embodiments, the application relates to the use of the pharmaceutical composition in the manufacture of a medicament for the treatment of a neurological or psychiatric disorder associated with glutamate dysfunction.

In certain embodiments, the application relates to a method for treating a neurological or psychiatric disorder associated with glutamate dysfunction, comprising administering to a subject in need thereof an effective amount of a compound formula (I) or (II), or an enantiomer thereof, or of a pharmaceutical composition comprising a compound of formula (I) or (II).

### DESCRIPTION OF THE FIGURES

FIG. 1 shows Compound A turnover in human hepatocytes in the presence or absence of a CYP3A inhibitor (Example 10). Approximately 10% of Compound A disappeared in 60 minutes. No turnover was observed when hepatocytes were treated with Compound A in the presence of CYP3A inhibitor ketoconazole (keto).

FIG. 2 shows Compound A metabolite formation in human hepatocytes in the presence or absence of CYP3A inhibitor from 0 minute to 60 minutes (Example 10). During this time period, the hydrolysis product (M346 metabolite) was not detected, while formation of the hydroxylation product (M438) and oxidative-ring-opening product (M453) was found to increase with incubation time and was inhibited when hepatocytes were treated with Compound A in the presence of CYP3A inhibitor ketoconazole (keto).

### DETAILED DESCRIPTION OF THE DISCLOSURE

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and neuroscience biology, and pharmacology, described herein, are those well known and commonly used in the art.

Chemistry terms used herein are used according to conventional usage in the art, for example as exemplified by “The McGraw-Hill Dictionary of Chemical Terms”, Parker S., Ed., McGraw-Hill, San Francisco, Calif. (1985).

TABLE 1

| | | |
|---|---|---|
| Compound | Structure | Name |

| | | |
|---|---|---|
| A | 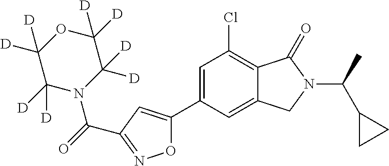 [SMILES: [2H]C1([2H])OC([2H])([2H])C([2H])([2H])N(C(=O)c2cc(-c3cc(Cl)c4c(c3)CN([C@@H](C)C3CC3)C4=O)on2)C1([2H])[2H]] | (S)-7-chloro-2-(1- cyclopropylethyl)-5-(3- (morpholine-4- carbonyl- 2,2,3,3,5,5,6,6- d₈)isoxazol-5- yl)isoindolin-1-one |
| B | 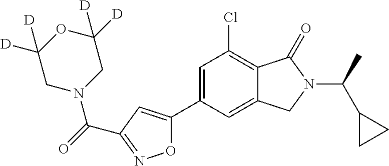 [SMILES: [2H]C1([2H])CN(C(=O)c2cc(-c3cc(Cl)c4c(c3)CN([C@@H](C)C3CC3)C4=O)on2)CC([2H])([2H])O1] | (S)-7-chloro-2-(1- cyclopropylethyl)-5-(3- (morpholine-4- carbonyl-2,2,6,6- d₄)isoxazol-5- yl)isoindolin-1-one |
| C | 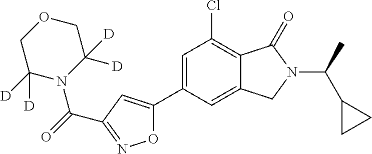 [SMILES: [2H]C1([2H])COCC([2H])([2H])N1C(=O)c1cc(-c2cc(Cl)c3c(c2)CN([C@@H](C)C2CC2)C3=O)on1] | (S)-7-chloro-2-(1- cyclopropylethyl)-5-(3- (morpholine-4- carbonyl-3,3,5,5- d₄)isoxazol-5- yl)isoindolin-1-one |
| D | 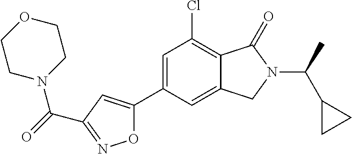 [SMILES: C[C@@H](C1CC1)N1Cc2cc(-c3cc(C(=O)N4CCOCC4)no3)cc(Cl)c2C1=O] | (S)-7-chloro-2-(1- cyclopropylethyl)-5-(3- (morpholine-4- carbonyl)isoxazol-5- yl)isoindolin-1-one |

As set forth, the present application provides deuterated compounds that exhibit activity as modulators of metabotropic glutamate receptors. As disclosed in WO 2009/148403, Compound D has favorable solubility, low capacity to activate the hERG ion channel, and is highly active in assays assessing mGluR2 modulator activity. Compounds B and C (see Table 1) showed a similar in vitro metabolic profile to that of Compound D (see Table 3), i.e., comparable stability and clearance in human liver microsomes and rat hepatocytes. However, the in vitro (Table 3) and in vivo (Table 3) profiles of Compound A were different compared to those of Compound D. Specifically, Compound A showed greater stability in human liver microsomes and rat hepatocytes. Compound A also showed reduced in vivo clearance in rats following intravenous administration. Moreover, under the assay conditions described in Example 8 (the CYP Isoform Phenotyping Assay), there was no measureable contribution of CYP3A4 for metabolizing Compound A. In comparison, Compound D was shown to be metabolized predominately by CYP3A4 under these conditions. However, as described in Example 10, when assays were conducted focusing on measuring the appearance of metabolites instead of the disappearance of the parent compound, formation of low levels of metabolites was observed for Compound A. Compounds A, B, C, and D exhibited comparable stability in dog hepatocytes (see Table 3).

### Compounds

In one aspect, the disclosure provides compounds of Formula (I):

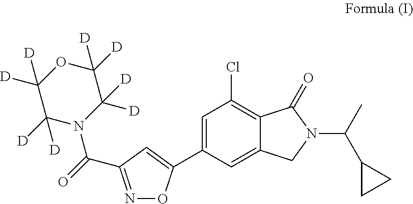 [SMILES: [2H]C1([2H])OC([2H])([2H])C([2H])([2H])N(C(=O)c2cc(-c3cc(Cl)c4c(c3)CN(C(C)C3CC3)C4=O)on2)C1([2H])[2H]]

or an enantiomer thereof.

In certain embodiments, the compound of Formula (I) is (S)-7-chloro-2-(1-cyclopropylethyl)-5-(3-(morpholine-4-carbonyl-2,2,3,3,5,5,6,6-d₈)isoxazol-5-yl)isoindolin-1-one, represented by Formula (II), represented by Formula (II):

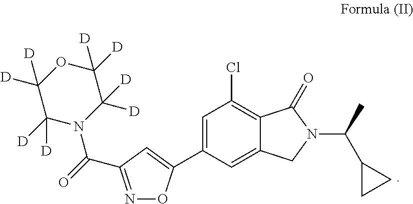 [SMILES: [2H]C1([2H])OC([2H])([2H])C([2H])([2H])N(C(=O)c2cc(-c3cc(Cl)c4c(c3)CN([C@@H](C)C3CC3)C4=O)on2)C1([2H])[2H]]

In certain embodiments, the disclosure provides compounds of Formula (III):

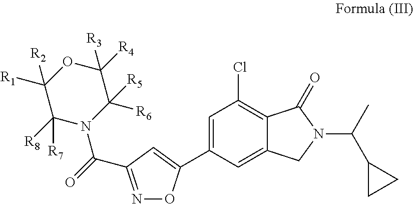

or an enantiomer or a diastereomer thereof, wherein
R₁-R₈ are each independently protium, deuterium or tritium, wherein at least two of R₁-R₈ are deuterium or tritium, such as deuterium.

In certain embodiments, the disclosure provides compounds of Formula (IV):

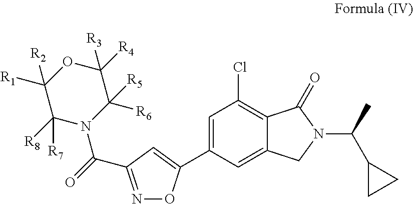

or an enantiomer or a diastereomer thereof, wherein
R₁-R₈ are each independently protium, deuterium or tritium, wherein at least two of R₁-R₈ are deuterium or tritium, such as deuterium.

In some embodiments wherein the compound of the disclosure is a compound of Formula (III), Formula (IV), or an enantiomer or diastereomer thereof, at least two of R₁-R₈ are deuterium. In certain such embodiments, the remaining R₁-R₈ are each protium.

In some embodiments wherein the compound of the disclosure is a compound of Formula (III), Formula (IV), or an enantiomer or diastereomer thereof, at least four of R₁-R₈ are deuterium. In certain such embodiments, the remaining R₁-R₈ are each protium.

In some embodiments wherein the compound of the disclosure is a compound of Formula (III), Formula (IV), or an enantiomer or diastereomer thereof, at least six of R₁-R₈ are deuterium. In certain such embodiments, the reamaingin R₁-R₈ are each protium.

In some embodiments wherein the compound of the disclosure is a compound of Formula (III), Formula (IV), or an enantiomer or diastereomer thereof, six of R₁-R₈ are deuterium. In certain such embodiments, the other two of R₁-R₈ are each protium.

In some embodiments wherein the compound of the disclosure is a compound of Formula (III), Formula (IV), or an enantiomer or diastereomer thereof, seven of R₁-R₈ are deuterium. In certain such embodiments, the other one of R₁-R₈ are each protium.

In some embodiments wherein the compound of the disclosure is a compound of Formula (III), Formula (IV), or an enantiomer or diastereomer thereof, all of R₁-R₈ are deuterium.

In some embodiments wherein the compound of the disclosure is a compound of Formula (III), Formula (IV), or an enantiomer or diastereomer thereof, at least two of R₁-R₄ are deuterium. In certain such embodiments, the remaining R₁-R₄ and R₅-R₈ are each protium.

In some embodiments wherein the compound of the disclosure is a compound of Formula (III), Formula (IV), or an enantiomer or diastereomer thereof, all of R₁-R₄ are deuterium. In certain such embodiments, R₅-R₈ are each protium.

In some embodiments wherein the compound of the disclosure is a compound of Formula (III), Formula (IV), or an enantiomer or diastereomer thereof, at least two of R₅-R₈ are deuterium. In certain such embodiments, the remaining R₅-R₈ and R₁-R₄ are each protium.

In some embodiments wherein the compound of the disclosure is a compound of Formula (III), Formula (IV), or an enantiomer or diastereomer thereof, all of R₅-R₈ are deuterium. In certain such embodiments, R₁-R₄ are each protium.

In some embodiments wherein the compound of the disclosure is a compound of Formula (III), Formula (IV), or an enantiomer or diastereomer thereof, at least two of R₁-R₄ and at least two of R₅-R₈ are deuterium. In certain such embodiments, the remaining R₁-R₈ are each protium.

In some embodiments wherein the compound of the disclosure is a compound of Formula (III), Formula (IV), or an enantiomer or diastereomer thereof, all of R₁-R₄ and at least two of R₅-R₈ are deuterium. In certain such embodiments, the remaining R₅-R₈ are each protium.

In some embodiments wherein the compound of the disclosure is a compound of Formula (III), Formula (IV), or an enantiomer or diastereomer thereof, at least two of R₁-R₄ and all of R₅-R₈ are deuterium. In certain such embodiments, the remaining R₁-R₄ are each protium.

In certain embodiments, the disclosure provides compounds of Formula (V):

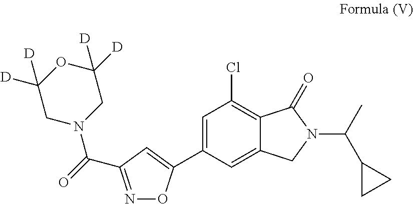 [SMILES: [2H]C1([2H])CN(C(=O)c2cc(-c3cc(Cl)c4c(c3)CN(C(C)C3CC3)C4=O)on2)CC([2H])([2H])O1]

or an enantiomer thereof.

In certain embodiments, the disclosure provides compounds of Formula (VI):

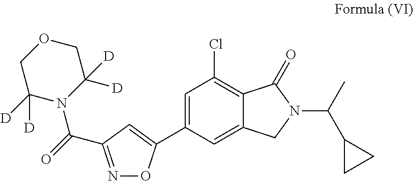 [SMILES: [2H]C1([2H])COCC([2H])([2H])N1C(=O)c1cc(-c2cc(Cl)c3c(c2)CN(C(C)C2CC2)C3=O)on1]

or an enantiomer thereof.

In certain embodiments, the disclosure provides the compound of Formula (VII):

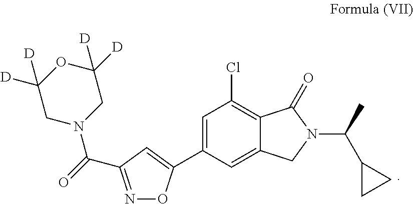 [SMILES: [2H]C1([2H])CN(C(=O)c2cc(-c3cc(Cl)c4c(c3)CN([C@@H](C)C3CC3)C4=O)on2)CC([2H])([2H])O1]

In certain embodiments, the disclosure provides the compound of Formula (VIII):

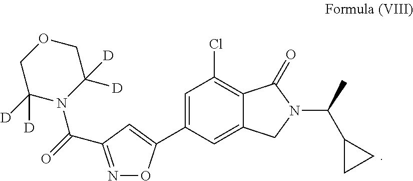 [SMILES: [2H]C1([2H])COCC([2H])([2H])N1C(=O)c1cc(-c2cc(Cl)c3c(c2)CN([C@@H](C)C2CC2)C3=O)on1]

In certain embodiments, the disclosure provides compounds of Formula (IX):

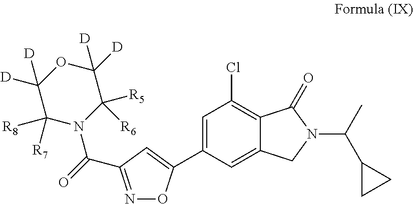

or an enantiomer or a diastereomer thereof, wherein
R₅-R₈ are each independently protium or tritium, such as protium.

In certain embodiments, the disclosure provides compounds of Formula (X):

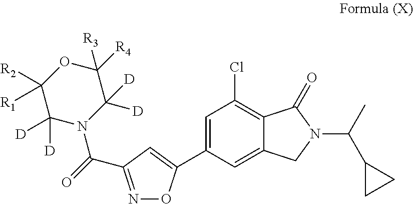

or an enantiomer or a diastereomer thereof, wherein
R₁-R₄ are each independently protium or tritium, such as protium.

In certain embodiments, the disclosure provides compounds of Formula (XI):

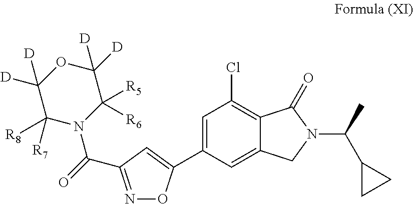

or an enantiomer or a diastereomer thereof, wherein
R₅-R₈ are each independently protium or tritium, such as protium.

In certain embodiments, the disclosure provides compounds of Formula (XII):

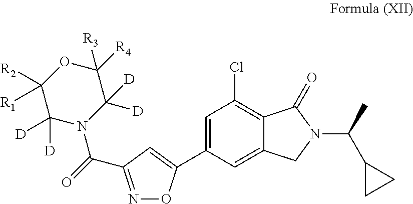

or an enantiomer or a diastereomer thereof, wherein R₁-R₄ are each independently protium or tritium, such as protium.

Compounds of the disclosure (e.g., compounds of formulae (I), (II), (III), (IV), (V), (VI), (VII), (VIII), (IX), (X), (XI), and (XII)) containing one or multiple asymmetrically substituted atoms may be isolated in optically active or racemic forms. It is well known in the art how to prepare optically active forms, such as by resolution of racemic forms, by synthesis from optically active starting materials, by synthesis using optically active reagents, or by asymmetric synthesis.

In certain embodiments, compounds of the disclosure may be racemic. In certain embodiments, compounds of the disclosure may be enriched in one enantiomer. For example, a compound of the disclosure may have greater than 30% ee, 40% ee, 50% ee, 60% ee, 70% ee, 80% ee, 90% ee, or even 95% or greater ee.

In certain embodiments, the therapeutic preparation may be enriched to provide predominantly one enantiomer of a compound (e.g., compounds of formulae (I), (II), (III), (IV), (V), (VI), (VII), (VIII), (IX), (X), (XI), and (XII)). An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2 mol percent of the second enantiomer.

In certain embodiments, compounds of the disclosure may have more than one stereocenter. In certain such embodiments, compounds of the disclosure may be enriched in one or more diastereomer. For example, a compound of the disclosure may have greater than 30% de, 40% de, 50% de, 60% de, 70% de, 80% de, 90% de, or even 95% or greater de.

In certain embodiments, the therapeutic preparation may be enriched to provide predominantly one diastereomer of a compound (e.g., compounds of formulae (III), (IV), (IX), (X), (XI) and (XII)). A diastereomerically enriched mixture may comprise, for example, at least 60 mol percent of one diastereomer, or more preferably at least 75, 90, 95, or even 99 mol percent.

A variety of compounds of the disclosure may exist in particular geometric or stereoisomeric forms. The compounds of the disclosure are understood to extend to, and embrace all such compounds, including tautomers, cis- and trans-isomers, R- and S-enantiomers, diastereomers, (D)-isomers, (L)-isomers, the racemic mixtures thereof, and other mixtures thereof, as being covered within the scope of this application. All tautomeric forms are encompassed in the present disclosure. Additional asymmetric carbon atoms may be present in a substituent such as an alkyl group. All such isomers, as well as mixtures thereof, are intended to be included in this application, unless the stereochemistry or isomeric form is specifically indicated.

The present application further includes all pharmaceutically acceptable isotopically labelled compounds (e.g., compounds of formulae (I), (II), (III), (IV), (V), (VI), (VII), (VIII), (IX), (X), (XI), and (XII), or enantiomers or diastereomers thereof). An “isotopically” or “radio-labelled” compound is a compound where one or more atoms are replaced or substituted by an atom having an atomic mass or mass number different from the atomic mass or mass number typically found in nature (i.e., naturally occurring). For example, in certain embodiments, in the compounds of the disclosure (e.g., compounds of formulae (I), (II), (III), (IV), (V), (VI), (VII), (VIII), (IX), (X), (XI), and (XII), or enantiomers or diastereomers thereof), hydrogen atoms are replaced or substituted by one or more deuterium or tritium.

Certain isotopically labelled compounds of the disclosure (e.g., compounds of formulae (I), (II), (III), (IV), (V), (VI), (VII), (VIII), (IX), (X), (XI), and (XII), or enantiomers or diastereomers thereof), for example, those incorporating a radioactive isotope, are useful in drug and/or substrate tissue distribution studies. The radioactive isotopes tritium, i.e., ³H, and carbon 14, i.e., ¹⁴C, are particularly useful for this purpose in view of their ease of incorporation and ready means of detection.

Substitution with heavier isotopes such as deuterium, i.e., ²H, provide certain therapeutic advantages resulting from greater metabolic stability, for example, increased in vivo half-life, reduced clearance, reduced dosage requirements, improved safety, or improved efficacy. Deuterated therapeutic agents may provide solutions to address important unmet medical needs (S. L. Harbeson, R. D. Tung, Medchem News, 2014, 2, 8-22).

Deuterium can be represented by the symbol D or ²H and is also known as heavy hydrogen. It is a stable, non-radioactive and naturally-occurring isotope of hydrogen. Deuterium has a mass of 2.014102 unified atomic mass unit (u) while protium or ¹H has a mass of 1.007825 u. An isotopic replacement (e.g., protium (¹H) by deuterium (D or ²H)) may result in a change in the rate of reaction. In many instances, that phenomenon is explained by the ground-state vibrational energy (called the zero-point vibrational energy) of the chemical bond between the two atoms. Heavier isotopes typically lower the ground-state vibrational energy, resulting in a higher activation energy barrier for bond cleavage, which means that more energy is required to break the bond. As deuterium is twice as heavy as protium, the resulting C-D bond is stronger than the corresponding C—H bond, thus, more energy is required to break the C-D bond than to break the C—H bond. Such a difference which may result in a rate change in the reaction is known as the deuterium isotope effect. The effect is particulary marked if the C—H bond is broken during the rate-determining step of a reaction, because substitution of protium with deuterium may result in a decrease in the reaction rate. The deuterium isotope effect can range from 1 (no isotope effect at all) to much larger numbers such as 24 or even 50 (See March's Advanced Organic Chemistry: Reactions, Mechanisms, and Structure, 5^{th} Edition by Michael B. Smith and Jerry March, page 297; and references therein).

Any atom in the compounds of the disclosure not specifically labelled as an isotope is meant to represent the given element at about its natural isotopic abundance. For example, H represents protium (¹H) with a natural abundance of 99.985% and deuterium (²H) with a natural abundance of 0.015%.

While the natural isotopic abundance may vary in a synthesized compound based on the reagents used in the synthesis, the concentration of naturally abundant stable hydrogen isotopes such as deuterium is negligible compared to the concentration of stable isotopic substitution in the compounds of the disclosure. Thus, when a particular position of the compounds of the disclosure contains a deuterium atom, the concentration of deuterium at that position is substantially greater than the natural abundance of deuterium, which is 0.015%. In some embodiments, a position containing a deuterium atom has a deuterium enrichment or deuterium incorporation or deuterium concentration of at least 1%, of at least 5%, of at least 10%, of at least 15%, of at least 20%, of at least 25%, of at least 30%, of at least 35%, of at least 40%, of at least 45%, of at least 50%, of at least 55%, of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, or of at least 99%.

The term “deuterium enrichment” refers to the percentage of incorporation of deuterium at a given position of the compounds of the disclosure in replacement of protium.

Substitutions with certain radioactive isotopes can be useful in drug and/or substrate tissue distribution studies. The radioactive isotopes tritium, i.e., ³H, and carbon 14, i.e., ¹⁴C, are particularly useful for this purpose in view of their ease of incorporation and ready means of detection.

Substitution with positron emitting isotopes, such as ¹¹C, ¹⁸F, ¹⁵O, and ¹³N, can be useful in Positron Emission Topography (PET) studies for examining substrate receptor occupancy.

Isotopically labelled compounds can generally be prepared by conventional techniques known to those skilled in the art or by processes analogous to those described in the accompanying examples using an appropriate isotopically labelled reagent in place of the non-labelled reagent previously employed. Suitable isotopes that may be incorporated in the compounds of the disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I.

One or more compounds of any of the above structures may be used in the manufacture of medicaments for the treatment of any diseases or conditions disclosed herein.

### Uses of the Compounds

In certain embodiments, the compounds of the disclosure exhibit activity as modulators of metabotropic glutamate receptors. In certain embodiments, the compounds of the disclosure exhibit activity as potentiators of the mGluR2 receptor. It is contemplated that the compounds of the disclosure will be useful in therapy as pharmaceuticals, in particular for the treatment of neurological or psychiatric disorders associated with glutamate dysfunction in an animal and particularly in a human.

More specifically, the neurological or psychiatric disorders associated with glutamate dysfunction include, but are not limited to, disorders such as cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance disorder, substance use disorder, substance withdrawal disorder, substance abuse disorder, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, and post-traumatic pain, tardive dyskinesia, sleep disorders, narcolepsy, attention deficit/hyperactivity disorder, and conduct disorder.

Compounds of the present application may be administered orally, parenterally, buccally, vaginally, rectally, by inhalation, by insufflation, sublingually, intramuscularly, subcutaneously, topically, intranasally, intraperitoneally, intrathoracically, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient. The quantity of the compound to be administered will vary for the patient being treated and will vary from about 100 ng/kg of body weight to 100 mg/kg of body weight per day. For instance, dosages can be readily ascertained by those skilled in the art from this disclosure and the knowledge in the art. This, the skilled artisan can readily determine the amount of compound and optional additives, vehicles, and/or carrier in compositions and to be administered in methods of the application.

In certain embodiments, the application relates to a compound according to formulae (I), (II), (III), (IV), (V), (VI), (VII), (VIII), (IX), (X), (XI), or (XII), or enantiomers or diastereomers thereof, or combinations of any of the foregoing compounds, for use as a medicament.

In certain embodiments, the application relates to the use of a compound according to formulae (I), (II), (III), (IV), (V), (VI), (VII), (VIII), (IX), (X), (XI), or (XII), or enantiomers or diastereomers thereof, or combinations of any of the foregoing compounds, in the treatment of a neurological or psychiatric disorder associated with glutamate dysfunction.

In certain embodiments, the application relates to the use of a compound according to formulae (I), (II), (III), (IV), (V), (VI), (VII), (VIII), (IX), (X), (XI), or (XII), or enantiomers or diastereomers thereof, or combinations of any of the foregoing compounds, in the manufacture of a medicament for the treatment of a neurological or psychiatric disorder associated with glutamate dysfunction.

In certain embodiments, the application relates to a pharmaceutical composition comprising a compound of formulae (I), (II), (III), (IV), (V), (VI), (VII), (VIII), (IX), (X), (XI), or (XII), or enantiomers or diastereomers thereof, or combinations of any of the foregoing compounds, for use in the treatment of a neurological or psychiatric disorder associated with glutamate dysfunction.

In certain embodiments, the application relates to a pharmaceutical composition comprising a compound of formulae (I), (II), (III), (IV), (V), (VI), (VII), (VIII), (IX), (X), (XI), or (XII), or enantiomers or diastereomers thereof, or combinations of any of the foregoing compounds, for use in the manufacture of a medicament for the treatment of a neurological or psychiatric disorder associated with glutamate dysfunction.

In certain embodiments, the application relates to a method for treating a neurological or psychiatric disorder associated with glutamate dysfunction comprising administering to a subject in need thereof an effective amount of a compound of formulae (I), (II), (III), (IV), (V), (VI), (VII), (VIII), (IX), (X), (XI), or (XII), or enantiomers or diastereomers thereof, or combinations of any of the foregoing compounds.

In certain embodiments, the application relates to a method for treating a neurological or psychiatric disorder associated with glutamate dysfunction comprising administering to a subject in need thereof an effective amount of a pharmaceutical composition comprising a compound of formulae (I), (II), (III), (IV), (V), (VI), (VII), (VIII), (IX), (X), (XI), or (XII), or enantiomers or diastereomers thereof, or combinations of any of the foregoing compounds.

In certain embodiments, the neurological or psychiatric disorder associated with glutamate dysfunction is selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance disorder, substance use disorder, substance withdrawal disorder, substance abuse disorder, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, and post-traumatic pain, tardive dyskinesia, sleep disorders, narcolepsy, attention deficit/hyperactivity disorder, and conduct disorder.

In certain embodiments, the neurological or psychiatric disorder is a substance use disorder. In certain embodiments, the substance use disorder comprises a substance withdrawal disorder or a substance abuse disorder. In certain such embodiments, the substance is nicotine.

In certain embodiments, the substance use disorder is a substance abuse disorder. In certain such embodiments, the substance is nicotine.

In certain embodiments, the neurological or psychiatric disorder is a substance abuse disorder. In certain such embodiments, the substance is nicotine.

In certain embodiments, the substance abuse disorder is an alcohol abuse disorder, a tobacco products abuse disorder, a cannabis abuse disorder, a stimulant abuse disorder, a depressant abuse disorder, a hallucinogen abuse disorder, or an opioid abuse disorder. In certain such embodiments, the substance is nicotine.

In certain embodiments, the substance abuse disorder is a tobacco products abuse disorder.

In certain embodiments, the substance use disorder is a substance withdrawal disorder. In certain such embodiments, the substance is nicotine.

In certain embodiments, the application relates to a compound of formulae (I), (II), (III), (IV), (V), (VI), (VII), (VIII), (IX), (X), (XI), or (XII), or enantiomers or diastereomers thereof formula I, II, III, IV, V, VI, VII, VIII, IX, X, XI and XII, for use in a treatment directed towards smoking cessation.

In certain embodiments, the application relates to a pharmaceutical composition comprising a compound of formulae (I), (II), (III), (IV), (V), (VI), (VII), (VIII), (IX), (X), (XI), or (XII), or enantiomers or diastereomers thereof, for use in a treatment directed towards smoking cessation.

In addition to their use in therapeutic medicine, one or more compounds of the disclosure are useful as pharmacological tools in the development and standardization of in vitro and in vivo test systems for the evaluation of the effects of potentiators of mGluR-related activity in laboratory animals as part of the search for new therapeutics agents. Such animals include, for example, cats, dogs, rabbits, monkeys, rats and mice.

In certain embodiments, one or more compounds of the disclosure may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same composition or in a separate composition, either simultaneously, sequentially, or by separate dosing of the individual components of the treatment. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of one or more compounds of the disclosure with one or more additional therapeutic agent(s) provides improved efficacy relative to each individual administration of the compound of the disclosure (e.g., a compound of formulae (I), (II), (III), (IV), (V), (VI), (VII), (VIII), (IX), (X), (XI), or (XII), or enantiomers or diastereomers thereof) or the one or more additional therapeutic agent(s). In certain such embodiments, the conjoint administration provides an additive effect, wherein an additive effect refers to the sum of each of the effects of individual administration of the compound of the disclosure and the one or more additional therapeutic agent(s).

One or more compounds of the disclosure may be administered concurrently, simultaneously, sequentially or separately with one or more therapeutic agents of the following categories of agents: nicotine replacement therapy (NRT) patches and gum, nicotinic receptor partial agonists, and certain antagonists of neuronal nicotinic acetylcholine receptors (nACh receptors). Such therapeutic agents may include one or more of the following agents: Wellbutrin (Wellbutrin XL oral, Wellbutrin oral, Wellbutrin SR oral), Chantix (Chantix oral, Chantix Continuing Month Pak Oral, Chantix Continuing Month Box Oral, Chantix Starting Month Pak Oral, Chantix Starting Month Box Oral), nortriptyline, clonidine clonidine HCl oral, clonidine transdermal), bupropion (bupropion HCl oral, bupropion HBr oral), Pamelor, Nicoderm CQ, Catapres (Catapres oral, Catapres-TT2 transdermal, Catapres-TT1 transdermal, Catapres-TT3 transdermal), Zyban, varenicline, Aplenzin, Buproban, Nicotrol (inhalation, NS nasal), Kapvay XL oral, Nicorette, nicotine ((polacrilex) buccal, nicotine nasal, nicotine transdermal, nicotine inhalation), Forfivo, Stop Smoking Aid buccal, Nicorelief buccal, NTS Step 1 transdermal, Quit (Quit 2 buccal, Quit 4 buccal), naltrexone (Vivitrol), methadone, buprenorphine, suboxone, naloxone (Narcan) and Campral.

Other therapeutic agents may include one or more of the following categories of agents:

(1) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine and equivalents and pharmaceutically active isomer(s) and metabolite(s) thereof;

(2) atypical antipsychotics including for example quetiapine and pharmaceutically active isomer(s) and metabolite(s) thereof;

(3) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;

(4) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(5) anticonvulsants including for example carbamazepine, valproate, lamotrogine, gabapentin and equivalents and pharmaceutically active isomer(s) and metabolite(s) thereof;

(6) Alzheimer's therapies including for example donepezil, memantine, tacrine and equivalents and pharmaceutically active isomer(s) and metabolite(s) thereof;

(7) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof;

(8) migraine therapies including for example almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(9) stroke therapies including for example abciximab, activase, NXY-059, citicoline, crobenetine, desmoteplase,repinotan, traxoprodil and equivalents and pharmaceutically active isomer(s) and metabolite(s) thereof;

(10) urinary incontinence therapies including for example darafenacin, falvoxate, oxybutynin, propiverine, robalzotan, solifenacin, tolterodine and equivalents and pharmaceutically active isomer(s) and metabolite(s) thereof;

(11) neuropathic pain therapies including for example gabapentin, lidoderm, pregablin and equivalents and pharmaceutically active isomer(s) and metabolite(s) thereof;

(12) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof;

(13) insomnia therapies including for example allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem and equivalents and pharmaceutically active isomer(s) and metabolite(s) thereof, or (14) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combination products employ one or more compounds of the disclosure within the dosage range described herein and the other pharmaceutically active compound or compounds within approved dosage ranges and/or the dosage described in the publication reference.

### Pharmaceutical Compositions and Methods of Administration

The disclosure further provides pharmaceutical compositions comprising one or more compounds of the disclosure together with a pharmaceutically acceptable carrier, excipient, or solvent. One or more of the compounds of the disclosure may be provided in isolated or purified form and/or as a pharmaceutical composition. One or more compounds of the disclosure may be formulated for administration in any convenient way for use in human medicine. Compounds or pharmaceutical compositions of the disclosure may be used in vitro or in vivo.

In certain embodiments, compositions of the disclosure include compounds provided as a pharmaceutical composition suitable for use in a human patient, comprising one or more of the compounds shown above (e.g., a compound of formulae (I), (II), (III), (IV), (V), (VI), (VII), (VIII), (IX), (X), (XI), or (XII), or enantiomers or diastereomers thereof), and one or more pharmaceutically acceptable carriers or excipients. In certain embodiments, the pharmaceutical composition comprises one or more compounds of formulae (I), (II), (III), (IV), (V), (VI), (VII), (VIII), (IX), (X), (XI), or (XII), or enantiomers or diastereomers thereof, and at least one pharmaceutically acceptable carrier or excipient. In certain embodiments, the pharmaceutical preparations may be for use in treating or preventing a condition or disease as described herein. In certain embodiments, the pharmaceutical preparations have a low enough pyrogen activity to be suitable for use in a human patient.

The term “pharmaceutical composition” refers to a composition suitable for pharmaceutical use in a subject animal, including humans and mammals, e.g., combined with one or more pharmaceutically acceptable carriers, excipients or solvents. Such a composition may also contain diluents, fillers, salts, buffers, stabilizers, solubilizers, and other materials well known in the art. In certain embodiments, a pharmaceutical composition encompasses a composition comprising the active ingredient(s), and the inert ingredient(s) that make up the excipient, carrier or diluent, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing one or more compounds of the disclosure and one or more pharmaceutically acceptable excipient(s), carrier(s) and/or diluent(s).

In certain embodiments, a “pharmaceutically acceptable” substance is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio. In certain embodiments, a “pharmaceutically acceptable” substance that is a component of a pharmaceutical composition is, in addition, compatible with the other ingredient(s) of the composition.

The terms “pharmaceutically acceptable excipient,” “pharmaceutically acceptable carrier” and “pharmaceutically acceptable diluent” encompass, without limitation, pharmaceutically acceptable inactive ingredients, materials, compositions and vehicles, such as liquid fillers, solid fillers, diluents, excipients, carriers, solvents and encapsulating materials. Carriers, diluents and excipients also include all pharmaceutically acceptable dispersion media, coatings, buffers, isotonic agents, stabilizers, absorption delaying agents, antimicrobial agents, antibacterial agents, antifungal agents, adjuvants, and so on. Each carrier must be “acceptable” in the sense of being compatible with the other ingredients of the composition and not injurious to the patient.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions. Except insofar as any conventional excipient, carrier or diluent is incompatible with the active ingredient, the present disclosure encompasses the use of conventional excipients, carriers and diluents in pharmaceutical compositions. See, e.g., Remington: The Science and Practice of Pharmacy, 21st Ed., Lippincott Williams & Wilkins (Philadelphia, Pa., 2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Preformulation and Formulation, Gibson, Ed., CRC Press LLC (Boca Raton, Fla., 2004).

A composition comprising a compound of the present disclosure may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

The characteristics of the carrier will depend on the route of administration. Each of the methods or uses of the present disclosure, as described herein, comprises administering to a mammal in need of such treatment or use an effective amount, such as a pharmaceutically or therapeutically effective amount, of one or more compounds of the disclosure, or a pharmaceutically acceptable salt thereof. Compounds of the disclosure may be administered alone or in combination with other agents.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Administering” or “administration of” one or more compounds or compositions of the disclosure to a subject can be carried out using one of a variety of methods known to those skilled in the art. For example, one or more compounds or compositions of the disclosure can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). One or more compounds or compositions of the disclosure can also appropriately be introduced by rechargeable or biodegradable polymeric devices or other devices, e.g., patches and pumps, or compositions, which provide for the extended, slow or controlled release of one or more compounds of the disclosure. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. When a method is part of a therapeutic regimen involving more than one compound, agent, substance, or treatment modality, the application contemplates that the agents may be administered at the same or differing times and via the same or differing routes of administration.

Appropriate methods of administering one or more compounds or compositions of the application to a subject will also depend, for example, on the age of the subject, whether the subject is active or inactive at the time of administering, whether the subject is cognitively impaired at the time of administering, the extent of the impairment, and the chemical and biological properties of the compound or composition (e.g., solubility, digestibility, bioavailability, stability and toxicity).

One or more compounds or pharmaceutical compositions of the application may be administered to cells in vitro, such as by addition to culture media. Additionally or alternatively, one or more compounds or pharmaceutical compositions of the application may be administered to route of administration, such as oral, parenteral, intravenous, intra-arterial, cutaneous, subcutaneous, intramuscular, topical, intracranial, intraorbital, ophthalmic, intravitreal, intraventricular, intracapsular, intraspinal, intracisternal, intraperitoneal, intranasal, aerosol, inhalation or insufflation (either through the mouth or the nose), mucosal, buccal, sublingual, transdermal, nasal, vaginal, rectal, central nervous system (CNS) administration, or administration by suppository. In some embodiments, the therapeutic methods of the application include administering the one or more compositions or compounds of the disclosure topically, systemically, or locally. One or more compositions or compounds described herein may be formulated as part of an implant or device, or formulated for slow or extended release. When administered parenterally, e.g., by intravenous, cutaneous or subcutaneous injection, the pharmaceutical compositions of one or more compounds for use in this application is preferably in a pyrogen-free, physiologically acceptable form.

In solid dosage forms for oral administration (capsules, tablets, pills, dragees, powders, cachets, lozenges, granules, and the like), one or more compositions comprising one or more compounds of the present application may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients. The tablet, capsule, and powder may contain from about 5 to 95% of one or more compounds of the present application, and preferably from about 10% to 90% of one or more compounds of the present application.

Liquid dosage forms for oral administration include pharmaceutically acceptable emulsions, microemulsions, solutions, suspensions, syrups, and elixirs. In addition to the compound of the present application, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents. When administered in liquid form, the pharmaceutical composition typically contains from about 0.5% to 90% by weight of one or more compounds of the present application, and preferably from about 1% to 50% by weight of one or more compounds of the present application.

Pharmaceutical compositions of the application for administration to the mouth may be presented as a mouthwash, or an oral spray, or an oral ointment.

Pharmaceutical compositions of the application for intravenous, cutaneous, or subcutaneous injection should contain, in addition to the toxicity-reducing compounds, an isotonic vehicle such as sodium chloride injection, Ringer's injection, dextrose injection, dextrose and sodium chloride injection, lactated Ringer's injection, or other vehicle as known in the art. The pharmaceutical composition of the one or more compounds of the present application may also contain stabilizers, preservatives, buffers, antioxidants, or other additives known to those of skill in the art. Techniques and compositions generally may be found in Remington's Pharmaceutical Sciences, Meade Publishing Co., Easton, Pa.

The pharmaceutical compositions of the application may be in the form of a liposome or micelles in which the toxicity-reducing compounds are combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal compositions include, without limitation, monoglycerides, diglycerides, sulfatides, lysolecithin, phospholipids, saponin, bile acids, and the like. Preparation of such liposomal compositions is within the level of skill in the art, as disclosed, for example, in U.S. Pat. Nos. 4,235,871; 4,501,728; 4,837,028; and 4,737,323, all of which are incorporated herein by reference.

Suspensions, in addition to one or more compounds of the disclosure may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Pharmaceutical compositions of the application for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Pharmaceutical compositions of the application which are suitable for vaginal administration also include pessaries, tampons, creams, gels, pastes, foams or spray compositions containing such carriers as are known in the art to be appropriate.

Alternatively or additionally, compositions of the application can be formulated for delivery via a catheter, stent, wire, or other intraluminal device. Delivery via such devices may be especially useful for delivery to the bladder, urethra, ureter, rectum, or intestine.

Dosage forms for the topical or transdermal administration include powders, sprays, ointments, pastes, creams, lotions, gels, solutions, patches and inhalants. One or more compounds of the disclosure may be mixed under sterile conditions with a pharmaceutically acceptable carrier, and with any preservatives, buffers, or propellants that may be required. The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to one or more compounds of the disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches provide controlled delivery of one or more compounds of the present application to the body. Such dosage forms can be made by dissolving or dispersing one or more compounds of the disclosure in the proper medium. Absorption enhancers can also be used to increase the flux of one or more compounds of the disclosure across the skin. The rate of such flux can be controlled by either providing a rate controlling membrane or dispersing the one or more compounds of the disclosure in a polymer matrix or gel.

Ophthalmic compositions, eye ointments, powders, solutions and the like, are also contemplated as being within the scope of this application. Exemplary ophthalmic compositions are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic compositions have properties similar to that of lacrimal fluids, aqueous humor or vitreous humor or are compatable with such fluids. A preferred route of administration is local administration (e.g., topical administration, such as eye drops, or administration via an implant).

The amount of compound(s) of the present application in the pharmaceutical composition will depend upon the nature and severity of the condition being treated, on the amount of the compound of the present application used, and on the nature of prior treatments the patient has undergone. Ultimately, the practitioner will decide the amount of the compound(s) of the present application with which to treat each individual patient. Representative doses of the present application include, but are not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during one day, especially when relatively large amounts are deemed to be needed. It is contemplated that the various pharmaceutical compositions used to practice the methods of the present application should contain about 0.1 μg to about 100 mg (preferably about 0.1 mg to about 50 mg, more preferably about 1 mg to about 2 mg) of compound per kg body weight.

The desired dose may conveniently be presented in a single dose or as divided doses administered at appropriate intervals, for example, as 2, 3, 4, or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

A “pharmaceutically acceptable salt” is a salt of a compound that is suitable for pharmaceutical use, including but not limited to metal salts (e.g., sodium, potassium, magnesium, calcium, etc.), acid addition salts (e.g., mineral acids, carboxylic acids, etc.), and base addition salts (e.g., ammonia, organic amines, etc.).

The phrase “pharmaceutically acceptable salt” or “salt” is used herein to refer to an agent or a compound according to the application that is a therapeutically active, non-toxic base and acid salt form of the compounds. The acid addition salt form of a compound that occurs in its free form as a base can be obtained by treating the free base form with an appropriate acid such as an inorganic acid. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzensulfonic, p-tolylsulfonic, citric, tartaric, methanesulfonic, camphorsulfonic and the like. See, e.g., WO 01/062726. Some pharmaceutically acceptable salts listed by Berge et al., Journal of Pharmaceutical Sciences, 66: 1-19 (1977), incorporated herein by reference in its entirety.

Compounds containing acidic protons may be converted into their therapeutically active, non-toxic base addition salt form, e.g., metal or amine salts, by treatment with appropriate organic and inorganic bases. Examples of pharmaceutically acceptable base addition salts include sodium, potassium, calcium, ammonium, organic amino, or magnesium salt, and the like, salts with organic bases, e. g. N-methyl-D-glucamine, hydrabamine salts, and salts with amino acids such as, for example, arginine, lysine and the like.

The neutral forms of the compounds of the disclosure are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the application.

In some embodiments, the compounds of the disclosure are in the form of a pharmaceutically acceptable salt. In some embodiments, the pharmaceutically acceptable salt is a hydrochloride, hydrobromide, phosphate, acetate, fumarate, maleate, tartrate, citrate, methanesulphonate or p-toluenesulphonate salt.

The term “solvate” refers to a compound of the disclosure or a pharmaceutically acceptable salt thereof, wherein molecules of suitable solvent are incorporated in a crystal lattice. A suitable solvent is physiologically tolerable at the dosage administered as the solvate.

The compounds of the disclosure, including their pharmaceutically acceptable salts, can also exist as various solvates, such as with water (also known as hydrates), methanol, ethanol, dimethylformamide, diethyl ether, acetamide, and the like, which are included within the scope of the present application. Such solvates include for example hydrates, alcoholates and the like. See, e.g., WO 01/062726. Mixtures of such solvates can also be prepared. The source of such solvate can be from the solvent of crystallization, inherent in the solvent of preparation or crystallization, or adventitious to such solvent.

The compounds of the disclosure, including their pharmaceutically acceptable salts, can also exist as various polymorphs, pseudo-polymorphs, or in amorphous state. As used herein, the term “polymorph” refers to different crystalline forms of the same compound and other solid state molecular forms including pseudo-polymorphs, such as hydrates, solvates, or salts of the same compound. Different crystalline polymorphs have different crystal structures due to a different packing of molecules in the lattice, as a result of changes in temperature, pressure, or variations in the crystallization process. Polymorphs differ from each other in their physical properties, such as x-ray diffraction characteristics, stability, melting points, solubility, or rates of dissolution in certain solvents. Thus, crystalline polymorphic forms are important aspects in the development of suitable dosage forms in pharmaceutical industry.

In certain embodiments, the application comprises a method for conducting a pharmaceutical business, by determining an appropriate composition and dosage of a compound of the disclosure for treating or preventing any of the diseases or conditions as described herein, conducting therapeutic profiling of identified compositions for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the application relates to a method for conducting a pharmaceutical business by determining an appropriate composition and dosage of one or more compounds of the disclosure for treating or preventing any of the disease or conditions as described herein, and licensing, to a third party, the rights for further development and sale of the composition.

This application will be better understood from the Experimental Details which follow. However, one skilled in the art will readily appreciate that the specific methods and results discussed are merely illustrative of the application as described more fully in the embodiments which follow thereafter.

### EXEMPLIFICATION

### Synthetic Methodology

Below follows a number of non-limiting examples of compounds of the disclosure. Compounds have been named using ChemBioDraw Professional 15.0.0.106.

The deuteration protocols infra are analogous to the procedure described within the article Cellulose, 2009, 16, 139-150 for the preparation of morpholine-d₈ hydrochloride.

### General Methods

All solvents used were of analytical grade and commercially available anhydrous solvents were routinely used for reactions. Starting materials used were available from commercial sources, or prepared according to literature procedures. Room temperature refers to 20-25° C. Solvent mixture compositions are given as volume percentages or volume ratios.

### NMR

NMR spectra were recorded on a 400-600 MHz NMR spectrometer fitted with a probe of suitable configuration. Spectra were recorded at ambient temperature unless otherwise stated. Chemical shifts are given in ppm down- and upfield from TMS (0.00 ppm). The following reference signals were used in ¹H-NMR: TMS δ 0.00, or the residual solvent signal of DMSO-d₆ δ 2.49, CD₃OD δ 3.30, acetone-d₆ 2.04 or CDCl₃ δ 7.26 (unless otherwise indicated). Resonance multiplicities are denoted s, d, t, q, m, br and app for singlet, doublet, triplet, quartet, multiplet, broad and apparent, respectively.

### TERMS AND ABBREVIATIONS

aq aqueous;
BnNH₂ dibenzylamine;
Cat. catalyst;
DCM dichloromethane;
DEA diethylamine;
DIPEA N,N-diisopropylethylamine
DMF N,N-dimethyl formamide;
DMSO dimethyl sulfoxide;
Et₂O diethyl ether;
EtOAc ethyl acetate;
EtOH ethanol;
eq. or equiv. equivalent;
h hour(s);
H₂O water;
H₂SO₄ sulfuric acid;
HPLC high performance liquid chromatography;
LCMS liquid chromatography mass spectrometry;
LiAlD₄ lithium aluminum deuteride;
LiAlH₄ lithium aluminum hydride;
MeOH methanol;
MeOD methanol-d₁;
min minute(s);
MS mass spectrometry;
NaH sodium hydride;
NaOH Sodium hydroxide;
NMR nuclear magnetic resonance;
Pd/C palladium on carbon or palladium on activated charcoal;
psi pounds per square inch;
rt or r.t. room temperature;
sat. saturated;
T3P propylphosphonic anhydride solution;
TFA trifluoroacetic acid;
THF tetrahydrofuran;
TLC thin layer chromatography;
TsCl tosyl chloride

Compounds A, B and C in Table 1 were prepared according to the synthetic schemes below.

### Example 1

### Preparation of (S)-7-chloro-2-(1-cyclopropylethyl)-5-(3-(morpholine-4-carbonyl-2,2,3,3,5,5,6,6-d₈)isoxazol-5-yl)isoindolin-1-one (Compound A, Scheme 1)

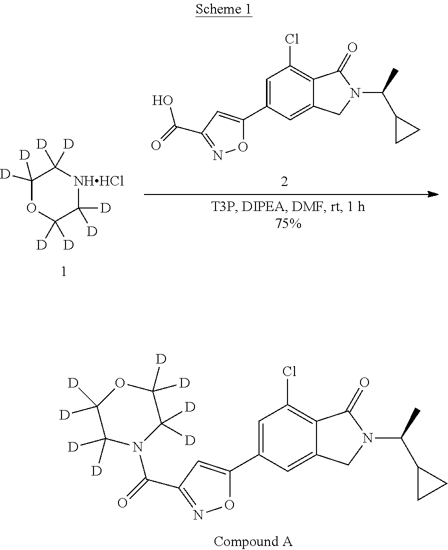

### Synthesis of (S)-7-chloro-2-(1-cyclopropylethyl)-5-(3-(morpholine-4-carbonyl-2,2,3,3,5,5,6,6-d₈)isoxazol-5-yl)isoindolin-1-one (Compound A)

1-Propylphosphonic acid cyclic anhydride solution (4.10 mL, 50 wt. % in N,N-dimethylformamide, 6.92 mmol) was added dropwise over 5 minutes to a clear yellow solution of (S)-5-(7-chloro-2-(1-cyclopropylethyl)-1-oxoisoindolin-5-yl)isoxazole-3-carboxylic acid (1.20 g, 3.46 mmol), morpholine-2,2,3,3,5,5,6,6-d₈ hydrochloride (1) (0.683 g, 5.19 mmol), and N,N-diisopropylethylamine (3.00 mL, 17.30 mmol) in anhydrous N,N-dimethylformamide (14.0 mL), and then stirred at room temperature for 1 hour. The reaction mixture was diluted with ethyl acetate (400 mL) and washed with saturated sodium bicarbonate (2×100 mL), saturated ammonium chloride (100 mL), and saturated sodium chloride (100 mL). The organic phase was dried over sodium sulfate, filtered, and concentrated under reduced pressure to provide (S)-7-chloro-2-(1-cyclopropylethyl)-5-(3-(morpholine-4-carbonyl-2,2,3,3,5,5,6,6-d8)isoxazol-5-yl)isoindolin-1-one 1 as a pale yellow solid (1.31 g). The impure material was purified by flash column chromatography (silica gel, 230-400 mesh, 150 g) eluting with 50-70% ethyl acetate/hexane. The fractions containing the product were concentrated to a gummy white solid, and further lyophilized to provide pure (S)-7-chloro-2-(1-cyclopropylethyl)-5-(3-(morpholine-4-carbonyl-2,2,3,3,5,5,6,6-d₈)isoxazol-5-yl)isoindolin-1-one (Compound A) as a fluffy white solid (1.12 g, 75%) with an HPLC purity of 98.63%.

¹H NMR (400 MHz, DMSO-d₆): 0.26 (1H, m), 0.41 (2H, m), 0.58 (1H, m), 1.15 (1H, m), 1.30 (3H, d), 3.58 (1H, m), 4.63 (2H, s), 7.56 (1H, s), 8.08 (1H, s), 8.12 (1H, s);

ES⁺ m/z [M+H]⁺ calcd for C₂₁H₁₅D₈ClN₃O₄⁺: 424; found: 424; HPLC Purity: 98.63% (BEH_C₁₈_{_}2.1×50 mm_1.7 μm).

### Example 2

### Preparation of (S)-7-chloro-2-(1-cyclopropylethyl)-5-(3-(morpholine-4-carbonyl-2,2,6,6-d₄)isoxazol-5-yl)isoindolin-1-one (Compound B, Scheme 2)

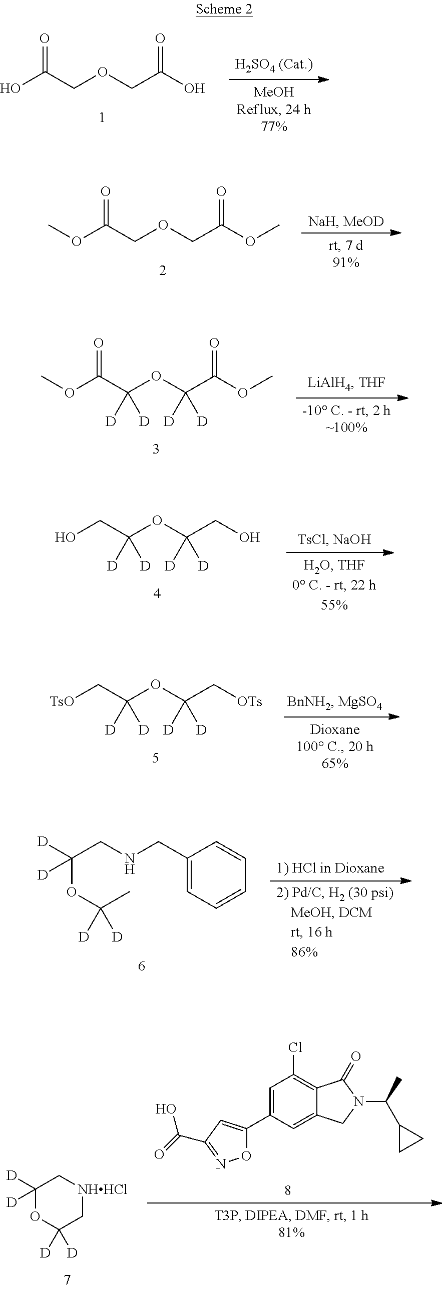

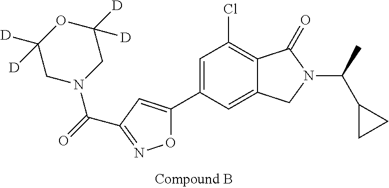 [SMILES: [2H]C1([2H])CN(C(=O)c2cc(-c3cc(Cl)c4c(c3)CN([C@@H](C)C3CC3)C4=O)on2)CC([2H])([2H])O1]

### Synthesis of dimethyl diglycolate (2)

Sulfuric acid (0.425 mL, 7.978 mmol) was added to a solution of diglycolic acid (1) (21.40 g. 159.59 mmol) in methanol (160 mL), and then stirred at reflux for 24 hours. The colorless solution was cooled to room temperature, and concentrated to a yellow oil. The crude material was dissolved in diethyl ether (750 mL), washed with water (250 mL), and saturated sodium chloride (250 mL). The organic phase was dried over sodium sulfate, filtered, and concentrated under reduced pressure to provide dimethyl diglycolate (2) as a white solid (19.95 g, 77%).

¹H NMR (400 MHz, CDCl₃): 3.78 (6H, s) 4.25 (4H, s); ES⁺ m/z [M+H]⁺ calcd for C₆H₁₁O₅⁺: 163; found: 163.

### Synthesis of dimethyl 2,2’-oxybis(acetate-d₄) (3)

Sodium hydride (493 mg, 60 wt. % dispersion in mineral oil, 12.34 mmol) was added slowly to methan(ol-d) (25.0 mL), and the cloudy solution was stirred under a nitrogen atmosphere for 5 minutes. Dimethyl diglycolate (2) (4.00 g 24.67 mmol) was added, and the mixture was further stirred under a nitrogen atmosphere at room temperature for 24 hours (˜73% exchanged by ¹H NMR).

The solvent was evaporated, fresh methan(ol-d) (25.0 mL) and sodium hydride (123 mg, 60 wt. % dispersion in mineral oil, 3.08 mmol) were added, and the resulting mixture was further stirred under a nitrogen atmosphere at room temperature for an additional 24 hours (˜89% exchanged by ¹H NMR).

The solvent was evaporated once again, fresh methan(ol-d) (25.0 mL) and sodium hydride (123 mg, 60wt. % dispersion in mineral oil, 3.08 mmol) were added, and the resulting mixture was further stirred under a nitrogen atmosphere at room temperature for an additional 24 hours (˜96% exchanged by ¹H NMR).

The solvent was evaporated for an additional time, fresh methan(ol-d) (25.0 mL) and sodium hydride (123 mg, 60wt. % dispersion in mineral oil, 3.08 mmol) were added, and the resulting mixture was further stirred under a nitrogen atmosphere at room temperature for 96 hours (˜99% exchanged by ¹H NMR). Finally, the solvent was evaporated for the last time, deuterium oxide (25.0 mL) was added, and the mixture was extracted with diethyl ether (3×100 mL). The combined organic phases were dried over sodium sulfate, filtered, and concentrated under reduced pressure to provide dimethyl 2,2′-oxybis(acetate-4) (3) as a colorless oil (3.71 g, 91%).

¹H NMR (400 MHz, CDCl₃): 3.78 (6H, s).

Synthesis of 2,2′-oxybis(ethan-2,2-d₂-1-ol) (4)

A solution of dimethyl 2,2′-oxybis(acetate-d₄) (3) (3.71 g, 22.33 mmol) in tetrahydrofuran (20 mL) was added to a cold (−10° C.) solution of lithium aluminum hydride (55.0 mL, 1M in tetrahydrofuran, 55.0 mmol). The reaction mixture was stirred, and allowed to warm to room temperature over 2 hours. The colorless reaction mixture was quenched with deionized water (12.50 mL), followed by aqueous 4.69M sodium hydroxide (12.50 mL 58.63 mmol), and deionized water (25.0 mL). The resultant suspension was filtered through a pad of Celite, the Celite pad was further washed with tetrahydrofuran (3×50 mL), and the filtrate was concentrated under reduced pressure, and then further lyophilized to provide 2,2′-oxybis(ethan-2,2-d₂-1-ol) as a colorless oil (4) (2.45 g, ˜100%). This material was used for the next step without further purification.

¹H NMR (400 MHz, CDCl₃): 3.77 (4H, s), 4.85 (2H, br s).

### Synthesis of oxybis(ethane-2,1-diyl-2,2-d₂) bis(p-toluenesulfonate) (5)

A solution of p-toluenesulfonyl chloride (9.33 g, 48.93 mmol) in tetrahydrofuran (25.0 mL) was added dropwise over 1 hour to a cold (0° C.) solution of 2,2′-oxybis(ethan-2,2-d₂-1-ol) (4) (2.45 g, 22.24 mmol) in aqueous 5.086 N sodium hydroxide (15.0 mL, 76.20 mmol), and then the mixture was further stirred as it gradually warmed to room temperature over 22 hours. Aqueous 1M hydrochloric acid (150 mL, 150 mmol) was added, and the mixture was stirred at room temperature for 1 hour. The resultant suspension was filtered, the collected material was rinsed with deionized water (3×20 mL) and hexane (20 mL), and then further dried under reduced pressure to provide oxybis(ethane-2,1-diyl-2,2-d₂) bis(p-toluenesulfonate) (5) as an off-white solid (5.13 g, 55%).

¹H NMR (400 MHz, CDCl₃): 2.45 (6H, s), 4.08 (4H, s), 7.35 (4H, d), 7.78 (4H, d). ES⁺ m/z [M+H]⁺ calcd for C₁₈H₁₉D₄O₇S₂⁺: 419; found: 419.

### Synthesis of 4-benzylmorpholine-2,2,6,6-d₄ (6)

Magnesium sulfate (11.10 g, 80.21 mmol) was added to a solution of oxybis(ethane-2,1-diyl-2,2-d₂) bis(p-toluenesulfonate) (5) (5.00 g, 11.95 mmol) and benzylamine (6.50 mL. 59.50 mmol) in 1,4-dioxane (120 mL), and then stirred at 100° C. for 20 hours. The reaction mixture was cooled to room temperature, the reaction mixture was filtered, and the filtrate was concentrated under reduced pressure to a cloudy yellow oil (4.77 g). The crude material was purified by flash column chromatography (silica gel, 230-400 mesh, 150 g) eluting with 10-50% ethyl acetate/hexane. The fractions containing the product were concentrated to provide 4-benzylmorpholine-2,2,6,6-d₄ (6) as a yellow oil (1.40 g, 65%).

¹H NMR (400 MHz, CDCl₃): 2.43 (4H, s), 3.50 (2H, s), 7.24-7.33 (5H, m); ES⁺ m/z [M+H]⁺ calcd for C₁₁H₁₂D₄NO⁺: 182; found: 182.

### Synthesis of morpholine-2,2,6,6-d₄ hydrochloride (7)

A solution of hydrogen chloride (19.0 mL, 4M in 1,4-dioxane, 76.0 mmol) was added to 4-benzylmorpholine-2,2,6,6-d₄ (6) (1.40 g (7.72 mmol). The mixture was sonicated for 5 minutes, and then concentrated to a white solid. The material was dissolved in a mixture of dichloromethane (25.0 mL) and methanol (50.0 mL), and transferred to a 500 mL Parr hydrogenation flask. Palladium on activated carbon (0.822 g, 10 wt. %, wet, Degussa type E101 NE/W, 0.772 mmol) was added, the flask was pressurized with hydrogen gas (30 psi), and shaken for 16 hours at room temperature. The reaction mixture was depressurized, filtered through Celite, and concentrated under reduced pressure to provide morpholine-2,2,6,6-d₄ hydrochloride (7) as a white solid (0.850 g, 86%).

¹H NMR (400 MHz, CD₃OD): 3.21 (4H, s); ¹³C NMR (100 MHz, CD₃OD): 43.1 (s), 62.8 (m); ES⁺ m/z [M+H]⁺ calcd for C₄H₆D₄NO⁺: 92; found: 92.

### Synthesis of (S)-7-chloro-2-(1-cyclopropylethyl)-5-(3-(morpholine-4-carbonyl-2,2,6,6-d4)isoxazol-5-yl)isoindolin-1-one (Compound B)

1-Propylphosphonic acid cyclic anhydride solution (2.60 mL, 50 wt. % in N,N-dimethylformamide, 4.33 mmol) was added dropwise over 3 minutes to a clear yellow solution of (S)-5-(7-chloro-2-(1-cyclopropylethyl)-1-oxoisoindolin-5-yl)isoxazole-3-carboxylic acid (8) (0.750 g, 2.16 mmol), morpholine-2,2,6,6-d₄ hydrochloride (7) (0.4.14 g, 3.24 mmol), and N,N-diisopropylethylamine (1.90 mL, 10.81 mmol) in anhydrous N,N-dimethylformamide (8.50 mL), and then stirred at room temperature for 1 hour. The reaction mixture was diluted with ethyl acetate (200 mL), washed with saturated sodium bicarbonate (2×50 mL), saturated ammonium chloride (50 mL), and saturated sodium chloride (50 mL). The organic phase was dried over sodium sulfate, filtered, and concentrated under reduced pressure to provide (S)-7-chloro-2-(1-cyclopropylethyl)-5-(3-(morpholine-4-carbonyl-2,2,6,6-d₄)isoxazol-5-yl)isoindolin-1-one 1 as a pale yellow solid (0.900 g). The impure material was purified by flash column chromatography (silica gel, 230-400 mesh, 100 g) eluting with 50-70% ethyl acetate/hexane. The fractions containing the product were concentrated to a gummy white solid, and further lyophilized to provide pure (S)-7-chloro-2-(1-cyclopropylethyl)-5-(3-(morpholine-4-carbonyl-2,2,6,6-d₄)isoxazol-5-yl)isoindolin-1-one (Compound B) as a fluffy white solid (0.750 g, 81%) with an HPLC purity of 97.63%.

¹H NMR (400 MHz, DMSO-d₆): 0.26 (1H, m), 0.41 (2H, m), 0.58 (1H, m), 1.16 (1H, m), 1.30 (3H, d), 3.58 (1H, m), 3.65 (2H, s), 3.67 (2H, s), 4.63 (2H, s), 7.56 (1H, s), 8.08 (1H, s), 8.12 (1H, s).

ES⁺ m/z [M+H]⁺ calcd for C₂₁H₁₉D₄ClN₃O₄⁺: 420; found: 420.

HPLC Purity: 97.63% (BEH_C₁₈_{_}2.1×50 mm_1.7 μm).

### Example 3

### Preparation of (S)-7-chloro-2-(1-cyclopropylethyl)-5-(3-(morpholine-4-carbonyl-3,3,5,5-d₄)isoxazol-5-yl)isoindolin-1-one (Compound C, Scheme 3)

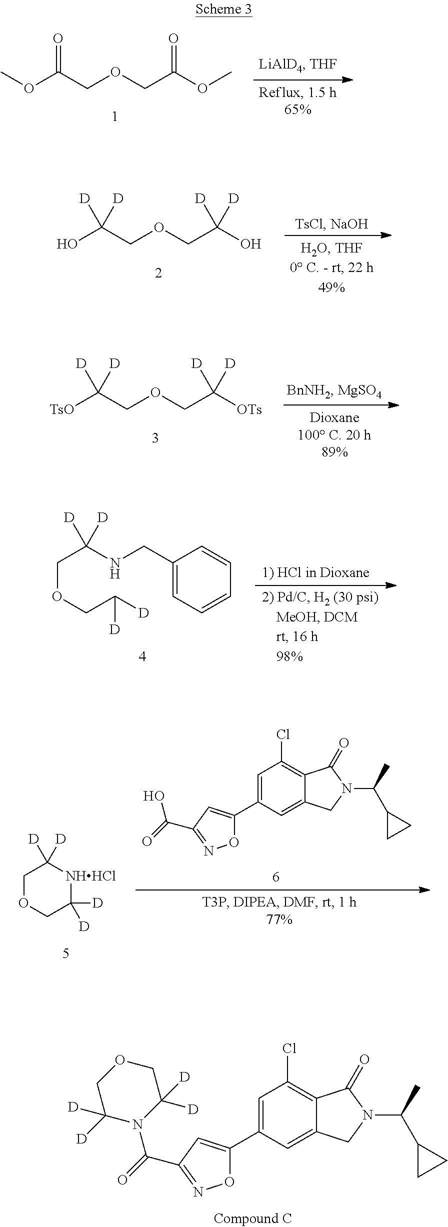

### Synthesis of 2,2′-oxybis(ethan-1,1-d₂-1-ol) (2)

A solution of dimethyl diglycolate (1) (7.50 g, 46.26 mmol) in tetrahydrofuran (80 mL) was added to a cold (−10° C.) suspension of lithium aluminum deuteride (4.79 g, 114.1 mmol) in tetrahydrofuran (160 mL). The reaction mixture was warmed to room temperature, and then stirred at reflux for 1.5 hours. The grey reaction mixture was quenched with deionized water (˜15 mL), and the resultant suspension was filtered through a pad of Celite. The Celite pad was further washed with tetrahydrofuran (3×50 mL), and the filtrate was concentrated under reduced pressure to provide 2,2′-oxybis(ethan-1,1-d₂-1-ol) as a colorless oil (2) (3.32 g, 65%). This material was used for the next step without further purification.

¹H NMR (400 MHz, CDCl₃): 3.61 (4H, s), 4.80 (2H, br s).

### Synthesis of oxybis(ethane-2,1-diyl-1,1-d₂) bis(p-toluenesulfonate) (3)

A solution of p-toluenesulfonyl chloride (12.64 g, 66.31 mmol) in tetrahydrofuran (30.0 mL) was added dropwise over 1 hour to a cold (0° C.) solution of 2,2′-oxybis(ethan-1,1-d₂-1-ol) (2) (3.32 g, 30.14 mmol) in aqueous 5.16 N sodium hydroxide (20.0 mL, 103.20 mmol), and then the mixture was further stirred as it gradually warmed to room temperature over 22 hours. Aqueous 1 M hydrochloric acid (200 mL, 200 mmol) was added, and the mixture was stirred at room temperature for 1 hour. The resultant suspension was filtered, the collected material was rinsed with deionized water (3×25 mL) and hexane (25 mL), and then further dried under reduced pressure to provide oxybis(ethane-2,1-diyl-1,1-d₂) bis(p-toluenesulfonate) (3) as an off-white solid (6.18 g, 49%).

¹H NMR (400 MHz, CDCl₃): 2.45 (6H, s), 3.60 (4H, s), 7.35 (4H, d), 7.78 (4H, d).

ES⁺ m/z [M+H]⁺ calcd for C₁₈H₁₉D₄O₇S₂⁻: 419; found: 419.

### Synthesis of 4-benzylmorpholine-3,3,5,5-d₄ (4)

Magnesium sulfate (11.10 g, 80.21 mmol) was added to a solution of oxybis(ethane-2,1-diyl-1,1-d₂) bis(p-toluenesulfonate) (3) (5.00 g, 11.95 mmol) and benzylamine (6.50 mL. 59.50 mmol) in 1,4-dioxane (120 mL), and then stirred at 100° C. for 20 hours. The reaction mixture was cooled to room temperature, the reaction mixture was filtered, and the filtrate was concentrated under reduced pressure to a cloudy yellow oil (5.73 g). The crude material was purified by flash column chromatography (silica gel, 230-400 mesh, 150 g) eluting with 10%-50% ethyl acetate/hexane. The fractions containing the product were concentrated to provide 4-benzylmorpholine-3,3,5,5-d₄ (4) as a yellow oil (1.93 g, 89%).

¹H NMR (400 MHz, CDCl₃): 3.50 (2H, s), 3.70 (4H, s), 7.24-7.33 (5H, m);

ES⁺ m/z [M+H]⁺ calcd for C₁₁H₁₂D₄NO⁺: 182; found: 182.

### Synthesis of morpholine-3,3,5,5-d₄ hydrochloride (5)

A solution of hydrogen chloride (26.0 mL, 4 M in 1,4-dioxane, 104 mmol) was added to 4-benzylmorpholine-3,3,5,5-d₄ (4) (1.93 g (10.65 mmol). The mixture was sonicated for 5 minutes, and then concentrated to a white solid. The material was dissolved in a mixture of dichloromethane (35.0 mL) and methanol (70.0 mL), and transferred to a 500 mL Parr hydrogenation flask. Palladium on activated carbon (1.13 g, 10 wt. %, wet, Degussa type E101 NE/W, 1.06 mmol) was added, the flask was pressurized with hydrogen gas (30 psi), and shaken for 16 hours at room temperature. The reaction mixture was depressurized, filtered through Celite, and concentrated under reduced pressure to provide morpholine-3,3,5,5-d₄ hydrochloride (5) as a white solid (1.33 g, 98%).

¹H NMR (400 MHz, CD₃OD): 3.87 (4H, s); ¹³C NMR (100 MHz, CD₃OD): 42.6 (m), 63.3 (s); ES⁺ m/z [M+H]⁺ calcd for C₄H₆D₄NO⁺: 92; found: 92.

### Synthesis of (S)-7-chloro-2-(1-cyclopropylethyl)-5-(3-(morpholine-4-carbonyl-3,3,5,5-d₄)isoxazol-5-yl)isoindolin-1-one (Compound C)

1-Propylphosphonic acid cyclic anhydride solution (2.60 mL, 50 wt. % in N,N-dimethylformamide, 4.33 mmol) was added dropwise over 3 minutes to a clear yellow solution of (S)-5-(7-chloro-2-(1-cyclopropylethyl)-1-oxoisoindolin-5-yl)isoxazole-3-carboxylic acid (6) (0.750 g, 2.16 mmol), morpholine-3,3,5,5-d₄ hydrochloride (5) (0.414 g, 3.24 mmol), and N,N-diisopropylethylamine (1.90 mL, 10.81 mmol) in anhydrous N,N-dimethylformamide (8.5 mL), and then stirred at room temperature for 1 hour. The reaction mixture was diluted with ethyl acetate (200 mL), washed with saturated sodium bicarbonate (2×50 mL), saturated ammonium chloride (50 mL), and saturated sodium chloride (50 mL). The organic phase was dried over sodium sulfate, filtered, and concentrated under reduced pressure to provide (S)-7-chloro-2-(1-cyclopropylethyl)-5-(3-(morpholine-4-carbonyl-3,3,5,5-d₄)isoxazol-5-yl)isoindolin-1-one 1 as a pale yellow solid (0.82 g). The impure material was purified by flash column chromatography (silica gel, 230-400 mesh, 100 g) eluting with 50-70% ethyl acetate/hexane. The fractions containing the product were concentrated to a gummy white solid, and further lyophilized to provide pure (S)-7-chloro-2-(1-cyclopropylethyl)-5-(3-(morpholine-4-carbonyl-3,3,5,5-d₄)isoxazol-5-yl)isoindolin-1-one (Compound C) as a fluffy white solid (0.710 g, 77%) with an HPLC purity of 98.44%.

¹H NMR (400 MHz, DMSO-d₆): 0.26 (1H, m), 0.41 (2H, m), 0.58 (1H, m), 1.16 (1H, m), 1.30 (3H, d), 3.58 (1H, m), 3.62 (2H, s), 3.68 (2H, s), 4.63 (2H, s), 7.56 (1H, s), 8.08 (1H, s), 8.12 (1H, s); ES⁺ m/z [M+H]⁺ calcd for C₂₁H₁₉D₄ClN₃O₄⁺: 420; found: 420; HPLC Purity: 98.44% (BEH_C₁₈_{_}2.1×50 mm_1.7 μm).

### Biological Activity

The pharmacological properties of the compounds of the disclosure can be analyzed using standard assays for functional activity. Examples of glutamate receptor assays are well known in the art as described in, for example, Aramori et al., 1992, Neuron, 8:757; Tanabe et al., 1992, Neuron, 8:169; Miller et al., 1995, J. Neuroscience, 15:6103; Balazs, et al., 1997, J. Neurochemistry, 1997,69:151. The methodology described in these publications is incorporated herein by reference. Conveniently, the compounds of the disclosure can be studied by means of an assay that measures the mobilization of intracellular calcium, [Ca²⁺]ᵢ in cells expressing mGluR2.

hERG activity was assessed using the process described by Bridgland-Taylor, M. H., et al, J. Pharm. Tox. Methods 54 (2006) 189-199.

Solubility was determined in pH 7.4 phosphate buffer after equilibration for 24 h at 25° C. and LC-MSMS were used for quantitation.

### Example 4

### Binding Assay (mGluR2 GTPγ³⁵S assay)

This assay was developed to measure the agonist and/or positive allosteric modulator (PAM) activity of compounds at the human metabotropic glutamate receptor 2 (mGluR2).

Crude membranes were prepared from CHO-K1 cells expressing human mGluR2 and stored at −80° C. in aliquots in 2 mg/mL concentrations using a previously described method Fenge et al., Cytotechnology 38 (2002). Assay buffer is defined as 0.22 μM filtered water containing 50 mM HEPES (Teknova), 10 mM MgCl₂ (Teknova), 50 mM NaCl (Teknova), 100 μM DTT (Sigma 43819), pH 7.4. Membrane solution containing membranes, beads, and GDP diluted in assay buffer was prepared in excess and incubated for 15 to 30 minutes at room temperature (25° C.). Membrane solution was prepared in a per-well ratio of 1.8 μg membranes, 225 μg PVT-WGA SPA beads (Perkin Elmer RPNQ0001), 3.3 μM GDP (Sigma G7127) in assay buffer to a final per-well volume of 180 μl. Stimulation buffer was prepared by diluting L-glutamate to 10 μM and GTPγ³⁵S to 1 nM in assay buffer. 2 μL of test compounds diluted in DMSO was delivered to individual wells of assay plates (Corning 3604). 180 μL of membranes solution was added to individual wells. 20 μL of stimulation buffer was then added to individual wells, plates were sealed, incubated on a shaker at room temperature for 45 minutes, centrifuged at 1000 RPM for 5 minutes and then assessed using a ³⁵S SPA Program (1 minute read time. Low Background Paralux) on a MicroBeta instrument (PerkinElmer, Waltham, Massachusetts). The EC₅₀ observed for Compound D was 121±56 nM and the EC₅₀ observed for Compound A was 170±9 nM.

### Example 5

### Metabolic Stability and Intrinsic Clearance in Human Liver Microsomes (HLM)

This assay was used to measure the in vitro metabolic stability of Compounds A, B, C and D of the application in pooled human liver microsomes. The concentration of parent compound in the reaction system for calculating intrinsic clearance of the compounds and estimating their stability in pooled human liver microsomes was evaluated by LC/MS/MS. 100 μmol/L solutions of each Compound A, B, C and D as well as 100 μmol/L solutions of each positive control compound (i.e., phenacetin, verapamil, diclofenac, imiprimine, benzydamine and metoprolol) were prepared by adding 2 μL of 10 mmol/L stock solution in DMSO to 198 μL of acetonitrile. A 1.1236 mg/mL HLM mixture was prepared by adding 1325 μL of 20 mg/mL HLM to 22260 μL of phosphate buffer. The HLM mixture (222.5 μL of the 1.1236 mg/mL mixture) and 25 μL of a 10 mM NADPH solution were added to the incubation plates. After mixing on a whirly mixer for 10 seconds, the incubation plates were pre-warmed at 37° C. for 8 minutes. The solutions of Compounds A, B, C and D (2.5 μL of the 100 μM solutions prepared before) as well as positive control solutions (2.5 μL of the 100 μM solutions prepared before) were added to the incubation plate. The incubation mixture was mixed on a whirly mixer for 10 seconds and incubation was carried out at 37° C. The reaction was quenched by transferring 20 μL of the incubated mixture at 0.5, 5, 10, 15, 20 and 30 minutes into the quenching plate containing 100 μL of cold acetonitrile. The quenching plates at 4000 rpm were centrifuged for 20 minutes, placed at 4 ° C. for 30 minutes, then re-centrifuged at 4000 rpm for 20 minutes to precipitate protein. The supernatant of each compound (40 μL) was transferred into a 96-well analysis plate. Compounds A, B, C and D were pooled together into one cassette, and pure water (160 μL) was added in each well. All incubations were performed in singlicate. API 4000 (AB sciex, USA) Ultra mass spectrometer was used to carry out the samples analysis in the MRM mode (MS/MS). Peak areas were determined from extracted ion chromatograms. Percentage of parent compound remaining was calculated from peak area of each compound or positive control. The slope value, k, was determined by linear regression of the natural logarithm of percent parent remaining vs. incubation time curve.

All calculations were carried out using Microsoft Excel. The in vitro half-life (in vitro t_{1/2}) was determined from the slope value, wherein in vitro _{t1/2}=−(0.693/k). Conversion of the in vitro_{t1/2} (in min) into the in vitro intrinsic clearance (in vitro CLᵢₙₜ, in μL/min/mg proteins) was done by using the following equation:

in vitro CLᵢₙₜ=(0.693/t_{1/2})*(volume of incubation (μL)/amount of proteins (mg)).

The results are presented in Table 3 infra.

### Example 6

### Metabolic Stability and Intrinsic Clearance in Rat Hepatocytes

Stock solutions (10 mM) of Compounds A, B, C, D and of control compounds were prepared in the appropriate solvent (DMSO). The L-15 Medium was placed in a 37° C. water bath, and allowed to warm for at least 15 minutes prior to use. Acetonitrile (80 μL) was added to each well of the 96-well plate (quenching plate).

In a new 96-well plate, the solutions of the above compounds and of the control compounds were diluted to 100 μM by combining 198 μL of acetonitrile and 2 μL of each stock solutions. A vial of cryopreserved rat hepatocytes was removed from storage, while ensuring that the vial remain at cryogenic temperatures until thawing process ensued. As quickly as possible, the cells were thawed by placing the vial in a 37° C. water bath and gently shaking the vial. The vial remained in the water bath until all ice crystals had dissolved and were no longer visible. After thawing was completed, the vial was sprayed with 70% ethanol, and transferred into a biosafety cabinet. The vial was opened and the content poured into a 50 mL conical tube containing L-15 Medium. The vial was washed twice. The 50 mL conical tube was placed in a centrifuge and spun at 50 g for 3 minutes (room temperature). The L-15 medium was aspirated. A small volume of buffer (˜200 μL) was added and the pellet was re-suspended. The tube was filled with buffer to a volume of 50 mL and centrifugation was repeated. Upon completion of spin, the L-15 Medium was aspirated and the hepatocytes re-suspended in enough incubation medium to yield ˜1.5×10⁶ cells/mL. Using Cellometer® Vision, the cells were counted and the viable cell density was determined. Cells with poor viability (<80% viability) were not acceptable for use. Cells were diluted with incubation medium to a working cell density of 1.0×10⁶ viable cells/mL. Hepatocytes (247.5 μL) were transferred into each well of a 96-well cell culture plate. The plate was placed on an Eppendorf Thermomixer Comfort plate shaker to allow the hepatocytes to warm for 10 minutes.

Solutions (2.5 μL of 100 μM) of Compounds A, B, C, D and of the control compounds were added into an incubation well containing cells and mixed to achieve a homogenous suspension at 0.5 minutes, which when achieved, defined the 0.5 minute time point. At the 0.5 minute time point, the incubated mixture (20 μL) was transferred to wells in a “Quenching plate,” followed by vortexing. The plate was incubated at 37° C. at 900 rpm on an Eppendorf Thermomixer Comfort plate shaker. At 5, 15, 30, 45, 60, 80, 100 and 120 minutes, the incubation system was mixed and samples (20 μL) of incubated mixture at each time point were transferred to wells in a separated “Quenching plate,” followed by vortexing. The quenching plates were centrifuged for 20 minutes at 4,000 rpm. Compounds A, B, C and D were pooled into one cassette and used for LC/MS/MS analysis. All incubations were performed in singlicate.

All calculations were carried out using Microsoft Excel. Peak areas were determined from extracted ion chromatograms. The in vitro half-life (t_{1/2}) of the parent compound was determined by regression analysis of the Ln percent parent disappearance vs. time curve.

The in vitro intrinsic clearance (in vitro Clᵢₙₜ, in μL/min/10⁶ cells) was determined from the slope value using the following equation:

in vitro Clᵢₙₜ=kV/N
V=incubation volume (0.25 mL);
N=number of hepatocytes per well (0.25×10⁶ cells).
The results are presented in Table 3 infra.

### Example 7

### Metabolic Stability and Intrinsic Clearance in Dog Hepatocytes

Stock solutions (10 mM) of the test compounds and control compounds were prepared in appropriate solvent (DMSO). The incubation medium (L-15 Medium) was placed in a 37° C. water bath, and warming was allowed for at least 15 minutes prior to use. 80 μL of acetonitrile was added to each well of the 96-well deep well plate (quenching plate). In a new 96-well plate, the 10 mM test compound solutions and the control compound solution were diluted to 100 μM by combining 198 μL of acetonitrile and 2 μL of 10 mM stock. A vial of cryopreserved dog hepatocytes was removed from storage, while ensuring that vials remain at cryogenic temperatures until thawing process ensued. As quickly as possible, the cells were thawed by placing the vial in a 37° C. water bath and gently shaking the vials. Vials remained in water bath until all ice crystals had dissolved and were no longer visible. After thawing was complete, vial was sprayed with 70% ethanol, and transferred to a biosafety cabinet. The vial was opened and the contents poured into the 50 mL conical tube containing thawing medium. The 50 mL conical tube was placed into a centrifuge and spun at 100 g for 10 minutes. Upon completion of spin, the thawing medium was aspirated and hepatocytes resuspended in enough incubation medium to yield ˜1.5×10⁶ cells/mL. Using Cellometer® Vision, cells were counted and the viable cell density was determined. Cells with poor viability (<80% viability) were not acceptable for use. Cells were diluted with incubation medium to a working cell density of 1.0×10⁶ viable cells/mL. 247.5 μL of hepatocytes were transferred into each well of a 96-well cell culture plate. The plate was placed on Eppendorf Thermomixer Comfort plate shaker to allow the hepatocytes to warm for 10 minutes. 2.5 μL of 100 μM test compound solution or control compound solution were added into an incubation well containing cells, and mixed to achieve a homogenous suspension at 0.5 minutes, which when achieved, defined the 0.5 minute time point. At the 0.5 minute time point, 20 μL of incubated mixture was transferred to wells in a “Quenching” plate followed by vortexing. The plate was incubated at 37 ° C. at 900 rpm on an Eppendorf Thermomixer Comfort plate shaker. At 5, 15, 30, 45, 60, 80, 100 minutes and 120 minutes, the incubation system was mixed and samples of 20 μL of incubated mixture were serially transferred at each time point to wells in a separate “Quenching” plate followed by vortexing. The quenching plates were centrifuged for 20 minutes at 4,000 rpm. Four different compounds were pooled into one cassette and used for LC/MS/MS analysis. All incubations were performed in duplicate.

All calculations were carried out using Microsoft Excel. Peak areas were determined from extracted ion chromatograms. Determine the in vitro intrinsic clearance (in vitro Clint, in μL/min/10⁶ cells) of parent compound by regression analysis of the Ln percent parent disappearance vs. time curve.

The in vitro intrinsic clearance (in vitro Clint, in μL/min/106 cells) is determined from the slope value using the following equation:

in vitro Clint=kV/N
V=incubation volume (0.25 mL);
N=number of hepatocytes per well (0.25×10⁶ cells).

The results are presented in Table 3 infra.

### Example 8

### CYP Isoform Phenotyping

This assay was used to determine whether Compounds A, B, C and D are metabolised by CYP enzymes.

Solutions (200 μmol/L) of each Compound A, B, C and D as well as of positive controls (phenacetin for CYP1A2, coumarin for CYP2A6, bupropion for CYP2B6, amitriptyline for CYP2C8, 2C19, 2D6 and 3A4, diclofenac for CYP2C9, chlorzoxazone for CYP2E1, midazolam for CYP3A5) were prepared by adding 4 μL of a 10 mmol/L stock solution in DMSO to 196 μL of acetonitrile. The recombinant human CYP isozymes were thawed on wet ice (over an hour). Mixtures (2670 μL) of CYP isozymes in phosphate buffer containing 112.36 nmol/L CYP1A2, 2A6, 2B6, 2C8, 2C9, 2C19, 2D6, 2E1, 3A4 or 3A5 were prepared. The CYP isozyme mixture (222.5 _{[}EL), 25 μL of 10 mmol/L of NADPH solution and 2.5 μL of vehicle control (2: 98 DMSO/acetonitrile) were added to the blank control plate. The resulting mixture was mixed on a whirly mixer for 10 seconds before a stop solution (1000 μL) with internal standard containing 200 nmol/L of 5,5-diethyl-1,3-diphenyl-2-iminobarbituric acid and 100 nmol/L of tolbutamide was added. The blank control plate was sealed with a lid and mixed on whirly mixer for 120 seconds, and kept on ice prior to centrifugation.

The standard samples containing 200 pmol/mL hydroxybupropion and 40 pmol/mL 6-hydroxychlorzoxazone were obtained as follows: the CYP isozyme mixture (222.5 μL) and 25 μL of 10 mmol/L NADPH solution were added to separate wells of the blank control plate. 2.5 μL of a 20 nmol/mL hydroxybupropion solution was added to one well and 2.5 μL of a 4 nmol/mL 6-hydroxychlorzoxazone solution was added to the other well. Mixing was conducted on a whirly mixer for 10 seconds. The stop solution with internal standard (1000 μL) was added to both wells. The blank control plate was sealed with a lid and mixing was conducted on a whirly mixer for 120 seconds. The blank control plate was kept on ice prior to centrifugation.

The CYP isozyme mixtures (222.5 μL) and Compounds A, B, C, D and the positive controls (2.5 μL of each 200 μmol/L solution) were added to the incubation plates. Mixing on a whirly mixer was conducted for 10 seconds. The incubation plate was pre-warmed at 37° C. for 15 minutes. The CYP/compound mixture (27 μL) was transferred from the incubation plate to the 0 minute “Quenching plates” containing 120 μL of the stop solution and 3 μL of a 10 mmol/L NADPH solution. The 0 minute “Quenching plates” was sealed with a lid and kept on ice. Mixing of the 0 minute “Quenching plates” on a whirly mixer was conducted for 120 seconds. The reaction was initiated with the addition of 22 μL of a 10 mmol/L of NADPH solution to the incubation plate. Mixing of the incubation mixture on a whirly mixer was conducted for 10 seconds. The incubation was carried out at 37° C. At 5, 10, 15 and 25 minutes, the incubation mixture was mixed on a whirly mixer for 10 seconds and samples of the incubated mixture (30 μL) were transferred at each time point to wells in a separate “Quenching plate” containing 120 μL of the stop solution, which was sealed with a lid and kept on ice. Mixing on a whirly mixer was conducted for 120 seconds. The quenching plates were centrifuged at 4000 rpm for 20 minutes, placed at 4° C. for 30 minutes, and re-centrifuged at 4000 rpm for 20 minutes to precipitate protein. The supernatant (80 μL) was quickly transferred to a separate “Analysis plate” for each above compounds and positive controls containing 80 μL of pure water for LC/MS/NIS analysis. The LC/MS/MS analysis was carried out on a Waters XEVO® TQD (mass spectrometer), with a UPLC-Pump (ACQUITY UPLC® 1-Class) and ACQUITY Sample Management FTN (autosampler). The UPLC-MS conditions are shown in Table 2.

TABLE 2

| Column | AQUITY UPLC ® BEH C18 1.7 μm 2.1*50 mm | | |
|---|---|---|---|
| Solvent A | Pure water + 0.1% Formic acid | | |
| Solvent B | Acetonitrile + 0.1% Formic acid | | |
| | Time | % | % |
| Gradient | (min) | A | B |
| | Initial | 95 | 5 |
| | 0.2 | 95 | 5 |
| | 1.0 | 0 | 100 |
| | 1.4 | 0 | 100 |
| | 1.6 | 95 | 5 |
| | 2.0 | 95 | 5 |

| | |
|---|---|
| Injection | 1 μL |
| Flow | 0.5 ml/mm |
| Split | No |
| Temperature | 40° C. |
| Run time | 2 min |
| Integration | Masslyns V4.1 |
| software | |
| MS | Capillary (kV): 3.00; Desolation Temp (° C.): 500; |
| Condition | Desolvation Gas Flow (L/Hr): 1000; Cone Gas Flow |
| | (L/Hr): 50 Cone voltage, product ion and collision |
| | energy are optimized manually. An optimization plate with |
| | solutions of the compounds, 1 μmol/L, in acetonitrile/water |
| | (50:50) is used. |

All calculations are carried out using Microsoft Excel. Peak areas were determined from extracted ion chromatograms. The percentage of parent remaining was calculated from peak area of Compounds A, B, C, D and positive controls. The slope value, k, was determined by linear regression of the natural logarithm of percent parent remaining vs. incubation time curve. The half life value (t_{1/2} rCYPi) was determined from the slope value: t_{1/2} rCYPi=0.693/k. Conversion of the t₁₁₂ rCYPi (in minutes) into the intrinsic clearance (CLint rCYPi in μL/min/pmol) was done using the following equation: CLint rCYPi=(0.693/t_{1/2})*(volume of incubation (μL)/amount of CYP (pmol)).

Degradation of bupropion (positive control for CYP2E1) and chlorzoxazone (positive control for CYP2B6) in the incubation systems was slight, and so concentrations of hydroxybupropion and 6-hydroxychlorzoxazone at 25 minutes in the incubation system were determined using the standard samples at single concentration and converted to the formation velocity of metabolites (pmol/min/nmol CYP) as below:

Metabolite Conc_{25 min sample}=(Peak Area_{25 min sample}/ Peak Area_{standard sample})* Metabolite Conc_{standard sample}

Formation Velocity=Metabolite Conc_{25 min sample} (pmol/mL)/(25 min*Conc_{CYP} (nmol CYP/mL))

The percentage contribution from an individual CYP isoform is calculated as follows:

$$\% {Contribution}_{CYPI} = \frac{{CL}_{intrCYPI} * {ISEF}_{rCYPI} * median abundance}{{\sum }_{i = 0}^{n} {CL}_{intrCYPI} * {ISEF}_{rCYPI} * median abundance} * 100 \%$$

The results are presented in Table 3 infra.

TABLE 3

| | Com- | Com- | Com- | Com- |
|---|---|---|---|---|
| | pound | pound | pound | pound |
| | A | B | C | D |
| Human Liver Microsomes | <3 | 9.5 | 7.0 | 9.5 |
| Median CLᵢₙₜ | | | | |
| (μL/min/mg) | | | | |
| Human Liver Microsomes | >231 | 73.0 | 99.6 | 73.2 |
| Median t_{1/2} (min) | | | | |
| Rat Hepatocytes | 6.9 | 18.8 | 23.8 | 18.8 |
| Median CLᵢₙₜ | | | | |
| (μL/min/mg) | | | | |
| Rat Hepatocytes | 100.4 | 36.8 | 29.1 | 37.3 |
| Median t_{1/2} (min) | | | | |
| Dog Hepatocytes | 17.3 | 17.3 | 20.1 | 23.2 |
| Median CLᵢₙₜ | | | | |
| (μL/min/mg) | | | | |
| Dog Hepatocytes | 40.1 | 40.1 | 33.3 | 29.9 |
| Median t_{1/2} (min) | | | | |
| CYP 3A4 | No | 82.3 | 99.7 | 100 |
| (% contribution) | measurable | | | |
| | contribution | | | |

### Example 9

### Pharmacokinetics

The animals were single dosed via intravenous (IV) bolus to tail vein over approximately 5 seconds. The animals were not fasted prior to dosing. The single dose formulation samples were collected from middle of formulation and stored at 5±3° C. for potential analysis. Animals were evaluated during in-life phase. The animals were dosed and the samples collected as shown in Table 4.

TABLE 4

| | | Treatment (IV) | | | |
|---|---|---|---|---|---|
| | No. of | Dosing | Dose | | |
| Animal | Animal | level | volume | Concentration | |
| Strain | (No/sex) | (mg/kg) | (mL/kg) | (mg/mL) | Route |
| Wistar | 2/male | 0.5 of each | 1 | 0.5 of each | IV |
| Rat/Harlan | | compound | | compound | |
| RCC | | | | | |

| | |
|---|---|
| Dosing | Dose Level: 0.5 mg/kg; Dose Volume: 1 mL/kg, |
| | IV bolus to tail vein over ~5 sec, n = 2 |
| Samples/ | Blood/via the tube cannulated in foot dorsal |
| Sampling Site | vein |
| Time Point(n = 2) | 2 minutes, 5 minutes, 10 minutes, 30 minutes, |
| | 1, 2, 4, 8, 24 hours post dose |
| Anticoagulant | EDTA |
| Volume/ | 0.2 mL (BLOOD) |
| Time point | |

The blood samples were centrifuged at 2000 g for 5 minutes at 4° C. to obtain plasma. Plasma samples were stored in polypropylene tubes, quickly frozen in ice box and kept at −80° C. Plasma samples were deprotainated by solvent precipitation. Concentration of Compounds A and D in plasma and tissue samples were analyzed using a LC-MS/MS method. WinNonlin version 6.2 will be used for pharmacokinetic parameters calculations. AUC was calculated using log trapezoidal method. Results for Compounds A and D are shown in Table 5.

TABLE 5

| | Compound A | Compound D |
|---|---|---|
| Nominal Dose (mg/kg) | 1.0 | 1.0 |
| CL (mL/min/kg) | 31.1 | 44.3 |
| t_{1/2} (h) | 0.761 | 0.813 |
| AUC (h*μmol/L) | 1.290 | 0.915 |

### Example 10

### CYP Assay in Human Hepatocytes to Identify Metabolite Formation

To identify the enzymes responsible for P450 mediated metabolism, the turnover of Compound A was tested in recombinant CYP enzymes (CYP1A2, 2A6, 2B6, 2C8, 2C9, 2C19, 2D6, 2E1, 3A4 and 3A5) at 0.1 μM and 1 μM. No significant turnover was observed in any of the the CYP enzymes tested. In contrast, Compound D was shown to be predominantly metabolised by CYP3A4 in this assay.

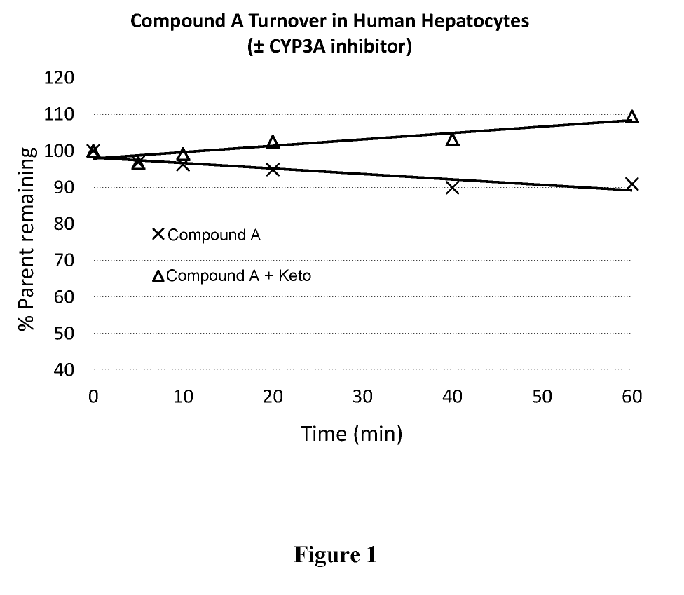

Since Compound D was predominantly metabolized by CYP3A4, further experiments were conducted with Compound A in human hepatocytes in the presence and absence of CYP3A inhibitor ketoconazole. As Compound A has very low turnover in human hepatocytes, both parent depletion and metabolite formation were monitored in these experiments. Parent disappearance data indicated a CYP3A involvement in the metabolism of Compound A (FIG. 1); however, the very low intrinsic clearance values make it difficult to provide an accurate determination of a percentage involvement of CYP3A via a substrate depletion method as described above.

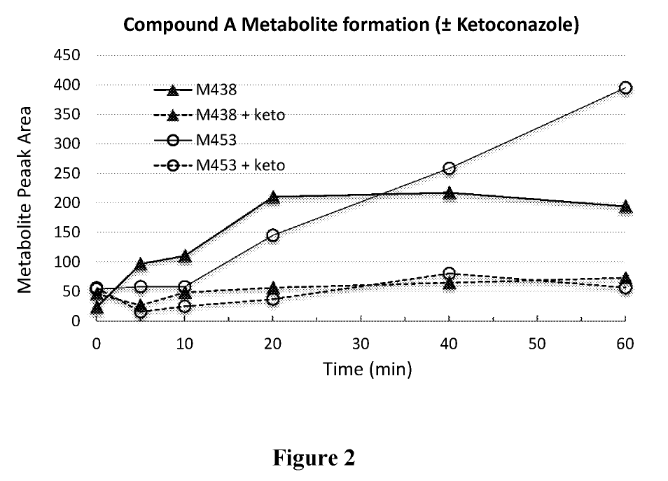

Within the same experiments, the formation of Compound A metabolites in the absence of ketoconazole was observed (FIG. 2). In comparison, a considerable drop in formation rates of the Compound A metabolites was observed in the presence of ketoconazole. The inhibition of metabolite formation by the addition of ketoconazole confirms the CYP3A contribution to the metabolism of Compound A. This data indicates that while CYP3A metabolism is still evident for Compound A, the rate of metabolism is significantly lower than that observed for the non-deuterated analogue Compound D.

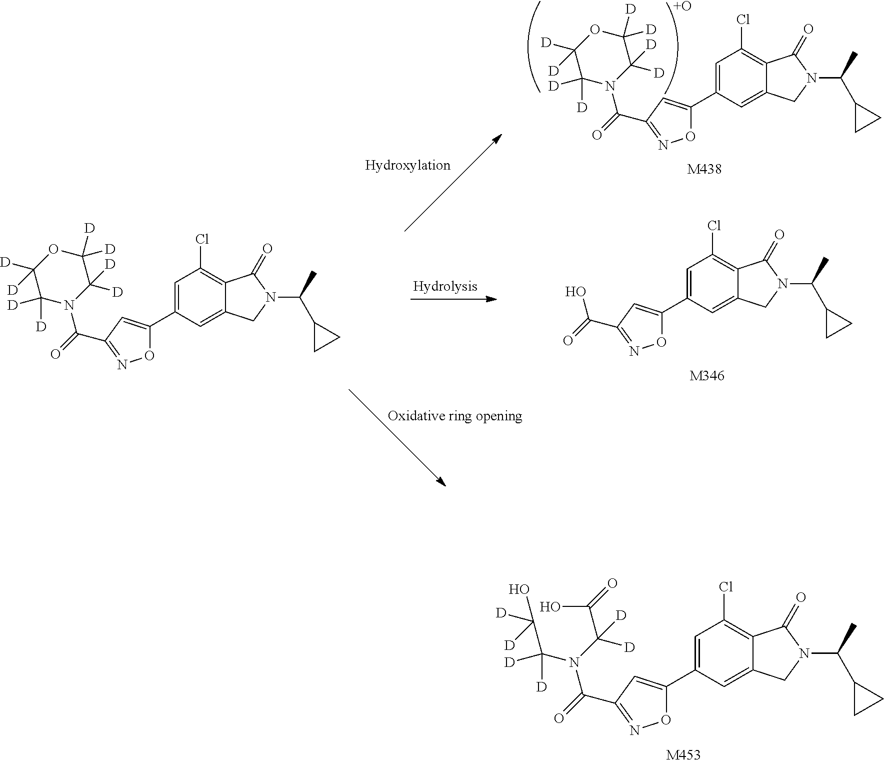

### Description of Method for Metabolism Identification and Relative Abundance Measurements

Compound A was incubated at 10 μM in human hepatocytes (1 million cells/mL) for 4 hours at 37° C. under air with 5% CO₂. The reaction was stopped by adding acetonitrile at a ratio of 3:1. Following centrifugation the supernatant was transferred to a clean tube and dried under a stream of N₂. Following reconstitution in 30% acetonitrile, aliquots of the sample were injected onto a LC/UV/MS system for metabolite identification and profiling. This allowed assessment of the metabolites formed and an indication of their relative abundance (via UV, Table 6).

TABLE 6

| Metabolite ID | Relative UV abundance (%) Human |
|---|---|
| M453 | 1.7 |
| M438 | 1.3 |
| M346 | 2.8 |
| Parent | 94.2 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject application have been discussed, the above specification is illustrative and not restrictive. Many variations of the subject of the application will become apparent to those skilled in the art upon review of this specification and the claims below. The full scope of the application should be determined by reference to the claims, along with their full scope of equivalents, and the specification, along with such variations.

1. A compound of Formula (I): 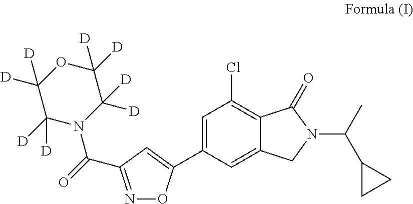 [SMILES: [2H]C1([2H])OC([2H])([2H])C([2H])([2H])N(C(=O)c2cc(-c3cc(Cl)c4c(c3)CN(C(C)C3CC3)C4=O)on2)C1([2H])[2H]]
or an enantiomer thereof.

2. The compound according to claim 1, wherein the compound is (S)-7-chloro-2-(1-cyclopropylethyl)-5-(3-(morpholine-4-carbonyl-2,2,3,3,5,5,6,6-d₈)isoxazol-5-yl)isoindolin-1-one, represented by Formula (II):

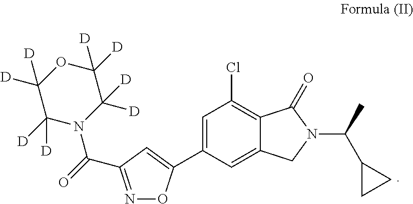 [SMILES: [2H]C1([2H])OC([2H])([2H])C([2H])([2H])N(C(=O)c2cc(-c3cc(Cl)c4c(c3)CN([C@@H](C)C3CC3)C4=O)on2)C1([2H])[2H]]

3. A pharmaceutical composition comprising as active ingredient a therapeutically effective amount of a compound according to claim 2, and at least one pharmaceutically acceptable carrier, excipient, or diluent.

4. A method for treating a neurological or psychiatric disorder associated with glutamate dysfunction, comprising administering to a subject in need thereof an effective amount of (S)-7-chloro-2-(1-cyclopropylethyl)-5-(3-(morpholine-4-carbonyl-2,2,3,3,5,5,6,6-d₈)isoxazol-5-yl)isoindolin-1-one, represented by Formula (II):

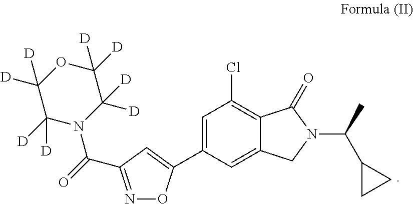 [SMILES: [2H]C1([2H])OC([2H])([2H])C([2H])([2H])N(C(=O)c2cc(-c3cc(Cl)c4c(c3)CN([C@@H](C)C3CC3)C4=O)on2)C1([2H])[2H]]

5. The method according to claim 4, wherein the neurological or psychiatric disorder is selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance disorder, substance use disorder, substance withdrawal disorder, substance abuse disorders, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, and post-traumatic pain, tardive dyskinesia, sleep disorders, narcolepsy, attention deficit/hyperactivity disorder, and conduct disorder.

6. The method according to claim 5, wherein the neurological or psychiatric disorder is a substance abuse disorder.

7. The method according to claim 6, wherein the substance abuse disorder is a tobacco products abuse disorder.

8. The method according to claim 6, wherein the substance is nicotine.

9. The method according to claim 5, wherein the neurological or psychiatric disorder is a substance withdrawal disorder.

10. The method according to claim 9, wherein the substance withdrawal disorder is a tobacco products withdrawal disorder.

11. The method according to claim 9, wherein the substance is nicotine.

12. The method according to claim 4, wherein the treatment is directed towards smoking cessation.

13. A method for treating a neurological or psychiatric disorder associated with glutamate dysfunction, comprising administering to a subject in need thereof an effective amount of a pharmaceutical composition; wherein the composition comprises as active ingredient a therapeutically effective amount of (S)-7-chloro-2-(1-cyclopropylethyl)-5-(3-(morpholine-4-carbonyl-2,2,3,3,5,5,6,6-d₈)isoxazol-5-yl)isoindolin-1-one, represented by Formula (II): 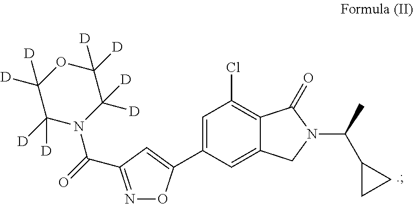 [SMILES: [2H]C1([2H])OC([2H])([2H])C([2H])([2H])N(C(=O)c2cc(-c3cc(Cl)c4c(c3)CN([C@@H](C)C3CC3)C4=O)on2)C1([2H])[2H]]
and at least one pharmaceutically acceptable carrier, excipient, or diluent.

14. The method according to claim 13, wherein the neurological or psychiatric disorder is selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance disorder, substance use disorder, substance withdrawal disorder, substance abuse disorders, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, and post-traumatic pain, tardive dyskinesia, sleep disorders, narcolepsy, attention deficit/hyperactivity disorder, and conduct disorder.

15. The method according to claim 14, wherein the neurological or psychiatric disorder is a substance abuse disorder.

16. The method according to claim 15, wherein the substance abuse disorder is a tobacco products abuse disorder.

17. The method according to claim 15, wherein the substance is nicotine.

18. The method according to claim 14, wherein the neurological or psychiatric disorder is a substance withdrawal disorder.

19. The method according to claim 18, wherein the substance withdrawal disorder is a tobacco products withdrawal disorder.

20. The method according to claim 18, wherein the substance is nicotine.

21. The method according to claim 13, wherein the treatment is directed towards smoking cessation.
